SEQUENCE LISTING (99 sequences)

SEQ ID NO: 1 - RNA, 20 nt, synthetic construct
  misc_feature 1..20: note = Synthetic sequence
  source 1..20: mol_type = other RNA
tatatctgggttgatggtac

SEQ ID NO: 2 - RNA, 20 nt, synthetic construct
  misc_feature 1..20: note = Synthetic sequence
  source 1..20: mol_type = other RNA
gccatgtatatctgggttga

SEQ ID NO: 3 - RNA, 20 nt, synthetic construct
  misc_feature 1..20: note = Synthetic sequence
  source 1..20: mol_type = other RNA
gcttgatgcctttgttcaag

SEQ ID NO: 4 - RNA, 20 nt, synthetic construct
  misc_feature 1..20: note = Synthetic sequence
  source 1..20: mol_type = other RNA
ggcctcctcgatgtgcctgg

SEQ ID NO: 5 - RNA, 20 nt, synthetic construct
  misc_feature 1..20: note = Synthetic sequence
  source 1..20: mol_type = other RNA
gcgctgcaaaacccgcaccc

SEQ ID NO: 6 - RNA, 20 nt, synthetic construct
  misc_feature 1..20: note = Synthetic sequence
  source 1..20: mol_type = other RNA
gtgtaaacggataatggaca

SEQ ID NO: 7 - RNA, 20 nt, synthetic construct
  misc_feature 1..20: note = Synthetic sequence
  source 1..20: mol_type = other RNA
gcattgtccaggcccccctt

SEQ ID NO: 8 - RNA, 20 nt, synthetic construct
  misc_feature 1..20: note = Synthetic sequence
  source 1..20: mol_type = other RNA
actgctgcaaaagagacgac

SEQ ID NO: 9 - RNA, 20 nt, synthetic construct
  misc_feature 1..20: note = Synthetic sequence
  source 1..20: mol_type = other RNA
gactggcttttagaatatgg

SEQ ID NO: 10 - RNA, 20 nt, synthetic construct
  misc_feature 1..20: note = Synthetic sequence
  source 1..20: mol_type = other RNA
actggcttttagaatatggt

SEQ ID NO: 11 - RNA, 20 nt, synthetic construct
  misc_feature 1..20: note = Synthetic sequence
  source 1..20: mol_type = other RNA
tttagaatatggtgggtttg

SEQ ID NO: 12 - RNA, 20 nt, synthetic construct
  misc_feature 1..20: note = Synthetic sequence
  source 1..20: mol_type = other RNA
caagtgggctatgcaaaagg

SEQ ID NO: 13 - RNA, 20 nt, synthetic construct
  misc_feature 1..20: note = Synthetic sequence
  source 1..20: mol_type = other RNA
tttccgcgatctgggatggc

SEQ ID NO: 14 - RNA, 20 nt, synthetic construct
  misc_feature 1..20: note = Synthetic sequence
  source 1..20: mol_type = other RNA
cgtggggcgtacaagttcca

SEQ ID NO: 15 - RNA, 20 nt, synthetic construct
  misc_feature 1..20: note = Synthetic sequence
  source 1..20: mol_type = other RNA
cgtaaaatctgctggtctcg

SEQ ID NO: 16 - RNA, 20 nt, synthetic construct
  misc_feature 1..20: note = Synthetic sequence
  source 1..20: mol_type = other RNA
gcttttgtacaaaaatgcat

SEQ ID NO: 17 - RNA, 20 nt, synthetic construct
  misc_feature 1..20: note = Synthetic sequence
  source 1..20: mol_type = other RNA
ccgagatgtcatgaaagaga

SEQ ID NO: 18 - RNA, 20 nt, synthetic construct
  misc_feature 1..20: note = Synthetic sequence
  source 1..20: mol_type = other RNA
catctctttcatgacatctc

SEQ ID NO: 19 - RNA, 20 nt, synthetic construct
  misc_feature 1..20: note = Synthetic sequence
  source 1..20: mol_type = other RNA
ccatctctttcatgacatct

SEQ ID NO: 20 - RNA, 20 nt, synthetic construct
  misc_feature 1..20: note = Synthetic sequence
  source 1..20: mol_type = other RNA
caagcggtacaaccccaaaa

SEQ ID NO: 21 - RNA, 20 nt, synthetic construct
  misc_feature 1..20: note = Synthetic sequence
  source 1..20: mol_type = other RNA
ggggttgtaccgcttgacca

SEQ ID NO: 22 - RNA, 20 nt, synthetic construct
  misc_feature 1..20: note = Synthetic sequence
  source 1..20: mol_type = other RNA
tattcctaatcactatgtcg

SEQ ID NO: 23 - RNA, 20 nt, synthetic construct
  misc_feature 1..20: note = Synthetic sequence
  source 1..20: mol_type = other RNA
gtacaaccccaaaatggtta

SEQ ID NO: 24 - RNA, 20 nt, synthetic construct
  misc_feature 1..20: note = Synthetic sequence
  source 1..20: mol_type = other RNA
aaaaagaccaagattaggtt

SEQ ID NO: 25 - RNA, 20 nt, synthetic construct
  misc_feature 1..20: note = Synthetic sequence
  source 1..20: mol_type = other RNA
cccttctccttcttacggga

SEQ ID NO: 26 - RNA, 20 nt, synthetic construct
  misc_feature 1..20: note = Synthetic sequence
  source 1..20: mol_type = other RNA
cagtcgacgacaaacccttc

SEQ ID NO: 27 - RNA, 20 nt, synthetic construct
  misc_feature 1..20: note = Synthetic sequence
  source 1..20: mol_type = other RNA
tattttcgatcatttgttcc

SEQ ID NO: 28 - RNA, 20 nt, synthetic construct
  misc_feature 1..20: note = Synthetic sequence
  source 1..20: mol_type = other RNA
gtttaaccctcgtacatccc

SEQ ID NO: 29 - RNA, 20 nt, synthetic construct
  misc_feature 1..20: note = Synthetic sequence
  source 1..20: mol_type = other RNA
aacaaatgatcgaaaatacc

SEQ ID NO: 30 - RNA, 20 nt, synthetic construct
  misc_feature 1..20: note = Synthetic sequence
  source 1..20: mol_type = other RNA
attttacttttataatagtc

SEQ ID NO: 31 - RNA, 20 nt, synthetic construct
  misc_feature 1..20: note = Synthetic sequence
  source 1..20: mol_type = other RNA
taatctctctgtctctcctt

SEQ ID NO: 32 - RNA, 20 nt, synthetic construct
  misc_feature 1..20: note = Synthetic sequence
  source 1..20: mol_type = other RNA
atattaggcacattcgttct

SEQ ID NO: 33 - RNA, 20 nt, synthetic construct
  misc_feature 1..20: note = Synthetic sequence
  source 1..20: mol_type = other RNA
agtaaaacatccccaggtcc

SEQ ID NO: 34 - RNA, 20 nt, synthetic construct
  misc_feature 1..20: note = Synthetic sequence
  source 1..20: mol_type = other RNA
caggtccaccaccatctgga

SEQ ID NO: 35 - RNA, 20 nt, synthetic construct
  misc_feature 1..20: note = Synthetic sequence
  source 1..20: mol_type = other RNA
ccggttccagatggtggtgg

SEQ ID NO: 36 - RNA, 20 nt, synthetic construct
  misc_feature 1..20: note = Synthetic sequence
  source 1..20: mol_type = other RNA
tctttacgtctacccatgca

SEQ ID NO: 37 - RNA, 20 nt, synthetic construct
  misc_feature 1..20: note = Synthetic sequence
  source 1..20: mol_type = other RNA
actctctttctttacgtcta

SEQ ID NO: 38 - RNA, 20 nt, synthetic construct
  misc_feature 1..20: note = Synthetic sequence
  source 1..20: mol_type = other RNA
tttcttctgttcttctcgtc

SEQ ID NO: 39 - RNA, 20 nt, synthetic construct
  misc_feature 1..20: note = Synthetic sequence
  source 1..20: mol_type = other RNA
aaacgagttctttttgttgt

SEQ ID NO: 40 - RNA, 20 nt, synthetic construct
  misc_feature 1..20: note = Synthetic sequence
  source 1..20: mol_type = other RNA
tgtgtcgtcttcgggttatc

SEQ ID NO: 41 - RNA, 20 nt, synthetic construct
  misc_feature 1..20: note = Synthetic sequence
  source 1..20: mol_type = other RNA
taagaatcgtcatgaacatc

SEQ ID NO: 42 - RNA, 20 nt, synthetic construct
  misc_feature 1..20: note = Synthetic sequence
  source 1..20: mol_type = other RNA
agactcacgtcggatgtccc

SEQ ID NO: 43 - RNA, 20 nt, synthetic construct
  misc_feature 1..20: note = Synthetic sequence
  source 1..20: mol_type = other RNA
tttctttaaccacttgaggg

SEQ ID NO: 44 - RNA, 20 nt, synthetic construct
  misc_feature 1..20: note = Synthetic sequence
  source 1..20: mol_type = other RNA
tcttgtgtcgagcagacggt

SEQ ID NO: 45 - RNA, 20 nt, synthetic construct
  misc_feature 1..20: note = Synthetic sequence
  source 1..20: mol_type = other RNA
agtagacgtagtcggtgtca

SEQ ID NO: 46 - RNA, 20 nt, synthetic construct
  misc_feature 1..20: note = Synthetic sequence
  source 1..20: mol_type = other RNA
gtgggttgtgaatggatgac

SEQ ID NO: 47 - RNA, 20 nt, synthetic construct
  misc_feature 1..20: note = Synthetic sequence
  source 1..20: mol_type = other RNA
ctgaggggtggtgcccgtcc

SEQ ID NO: 48 - RNA, 20 nt, synthetic construct
  misc_feature 1..20: note = Synthetic sequence
  source 1..20: mol_type = other RNA
gaccaagggtgtggttgtgc

SEQ ID NO: 49 - RNA, 20 nt, synthetic construct
  misc_feature 1..20: note = Synthetic sequence
  source 1..20: mol_type = other RNA
caaaacctaatggagtctgg

SEQ ID NO: 50 - RNA, 20 nt, synthetic construct
  misc_feature 1..20: note = Synthetic sequence
  source 1..20: mol_type = other RNA
atagatggagcacaaccaca

SEQ ID NO: 51 - RNA, 20 nt, synthetic construct
  misc_feature 1..20: note = Synthetic sequence
  source 1..20: mol_type = other RNA
tctgtaacaactgaagggtc

SEQ ID NO: 52 - RNA, 20 nt, synthetic construct
  misc_feature 1..20: note = Synthetic sequence
  source 1..20: mol_type = other RNA
ggaaaaacttgtctttgtct

SEQ ID NO: 53 - RNA, 20 nt, synthetic construct
  misc_feature 1..20: note = Synthetic sequence
  source 1..20: mol_type = other RNA
atatggttcaactctgtcct

SEQ ID NO: 54 - RNA, 20 nt, synthetic construct
  misc_feature 1..20: note = Synthetic sequence
  source 1..20: mol_type = other RNA
ttattgactctagtttctac

SEQ ID NO: 55 - RNA, 20 nt, synthetic construct
  misc_feature 1..20: note = Synthetic sequence
  source 1..20: mol_type = other RNA
agatcgttacaacacagtcc

SEQ ID NO: 56 - RNA, 20 nt, synthetic construct
  misc_feature 1..20: note = Synthetic sequence
  source 1..20: mol_type = other RNA
cgttacaacacagtccacct

SEQ ID NO: 57 - RNA, 20 nt, synthetic construct
  misc_feature 1..20: note = Synthetic sequence
  source 1..20: mol_type = other RNA
cgaaagaaagagtcctgttg

SEQ ID NO: 58 - RNA, 20 nt, synthetic construct
  misc_feature 1..20: note = Synthetic sequence
  source 1..20: mol_type = other RNA
ataatgaatgcacttttggc

SEQ ID NO: 59 - RNA, 20 nt, synthetic construct
  misc_feature 1..20: note = Synthetic sequence
  source 1..20: mol_type = other RNA
agactttcaagggtttgagc

SEQ ID NO: 60 - RNA, 20 nt, synthetic construct
  misc_feature 1..20: note = Synthetic sequence
  source 1..20: mol_type = other RNA
gacaatccatctattggaca

SEQ ID NO: 61 - RNA, 20 nt, synthetic construct
  misc_feature 1..20: note = Synthetic sequence
  source 1..20: mol_type = other RNA
tgacaatccatctattggac

SEQ ID NO: 62 - RNA, 20 nt, synthetic construct
  misc_feature 1..20: note = Synthetic sequence
  source 1..20: mol_type = other RNA
atccatctattggacaccct

SEQ ID NO: 63 - RNA, 20 nt, synthetic construct
  misc_feature 1..20: note = Synthetic sequence
  source 1..20: mol_type = other RNA
ggtccgcccttaggggggtc

SEQ ID NO: 64 - RNA, 20 nt, synthetic construct
  misc_feature 1..20: note = Synthetic sequence
  source 1..20: mol_type = other RNA
aaatttcgaacgtttggtcc

SEQ ID NO: 65 - RNA, 20 nt, synthetic construct
  misc_feature 1..20: note = Synthetic sequence
  source 1..20: mol_type = other RNA
ccgtgatacatgactaggtg

SEQ ID NO: 66 - RNA, 20 nt, synthetic construct
  misc_feature 1..20: note = Synthetic sequence
  source 1..20: mol_type = other RNA
cctctgtcggctcttatccc

SEQ ID NO: 67 - RNA, 20 nt, synthetic construct
  misc_feature 1..20: note = Synthetic sequence
  source 1..20: mol_type = other RNA
tcaccgaacatgaccagtgc

SEQ ID NO: 68 - RNA, 20 nt, synthetic construct
  misc_feature 1..20: note = Synthetic sequence
  source 1..20: mol_type = other RNA
gcactggtcatgttcggtga

SEQ ID NO: 69 - RNA, 20 nt, synthetic construct
  misc_feature 1..20: note = Synthetic sequence
  source 1..20: mol_type = other RNA
gtttcagtttccatttatgt

SEQ ID NO: 70 - RNA, 20 nt, synthetic construct
  misc_feature 1..20: note = Synthetic sequence
  source 1..20: mol_type = other RNA
ggtttcgtactattttctcc

SEQ ID NO: 71 - RNA, 20 nt, synthetic construct
  misc_feature 1..20: note = Synthetic sequence
  source 1..20: mol_type = other RNA
agtgaacacttatcgaatgt

SEQ ID NO: 72 - RNA, 20 nt, synthetic construct
  misc_feature 1..20: note = Synthetic sequence
  source 1..20: mol_type = other RNA
ggttttcatcacaatcctgt

SEQ ID NO: 73 - RNA, 20 nt, synthetic construct
  misc_feature 1..20: note = Synthetic sequence
  source 1..20: mol_type = other RNA
tataggaacaaaggtatgac

SEQ ID NO: 74 - RNA, 20 nt, synthetic construct
  misc_feature 1..20: note = Synthetic sequence
  source 1..20: mol_type = other RNA
gactggagtcatacctttgt

SEQ ID NO: 75 - RNA, 20 nt, synthetic construct
  misc_feature 1..20: note = Synthetic sequence
  source 1..20: mol_type = other RNA
tatttttaaccattgttcgc

SEQ ID NO: 76 - RNA, 20 nt, synthetic construct
  misc_feature 1..20: note = Synthetic sequence
  source 1..20: mol_type = other RNA
tttatttttaaccattgttc

SEQ ID NO: 77 - RNA, 20 nt, synthetic construct
  misc_feature 1..20: note = Synthetic sequence
  source 1..20: mol_type = other RNA
gaaccctcggtctccgtcca

SEQ ID NO: 78 - DNA, 4465 bp, Cricetulus sp.
  source 1..4465: mol_type = genomic DNA
tttgctttaccagtttcaaattacaatgagaagcctcttctttcccagcagggttgtgcttacatttctccttagatctctcttgaagagggctggtatatttgtgcctgctggaggtggaattaactgtaagaaggaggaaaagattgaatggatttacaggaaggatttcaagtaaactcagggaaacacatttacttgaacagtataaccctgggtcttgttttacactaagaccatacaagagatgttcgagttatcactagactgccggacaaatagaattccaacagcaaggtgcagatcagcgtagatctgtggttctgacctcggaatttgtcttggaaggagttcaagcactgagaagaagtcagagccagtgaagagaacttgggagccacagtctatcagaagaccagcttttcgtcagttcaaataccaaaggcctgattatcataaattcacataggaatgcataggttatttgatcaaggaattttagccagcttttactacatcaatactgcaaaagttcttaacaactgtggataattagaaatctgaatttcagctttcatagaaataactactcttgacgtattttaaaatatttacaacaggagaagaaaatggagtgaggatgttgtgttcagaaataccactgtcatgaagaataggtaaatttgtttttacaactttacagctactggcacagtggacctccaaggaacctggattattattattatttttaagtggacaagaaggattaaacataccgacttttgcttttgtacaaaaatgcattggactgtgtttttccttaggggtattgtgggcttcctctggagcggctgggttcaagtgggctatgcaaaaggaggtttgggagacaatcatgtccactccagttttatttatagaagactgcggaaccatgaaagacgggaaatacagagggaaattctctctattttgggtttgcctcacagacccaggccattttcacctggaaaacaagcatcttctgcacccctctttatgctggatctgtacaatgccatggccagcgaggaaaatcctgaagagtcggaatacttggtgagggtatccctggcaggggaggccaaagaggcaagaaagggatacccagcctctcccaatgggtatgctcatcgtatgcagttatctccaaggattcctctgactacccagagccctcctcttgccagcctacatgataccaactttctgaatgatgctgacatggtcatgagctttgtcaacttagttgaaagagacaaggacttttctcatcagaggaggcattacaaagaatttcgatttgatctgactcagattccacacggagaagcagtgacagcagctgaattccggatatataaggacaagagccaccaacgatttgaaaatgagacaattaagattagcatatatcagatcatcaaggaatacacaaatagggatgccgatctttttctgttagacacaaggaaggcacaagctttggatgtgggttggcttgtctttgacatcactgtgaccagcaaccattgggtgattaacccacagaacaatttgggcttacagctctgtgcagagacgggggatggacgcagtattaacgtaaaatctgctggtctcgtgggaagacatggacctcagtcaaaacagccattcatggtggccttcttcaaggcaagtgaggtacttcttcgatctgtgagagcagctaacaaacggaaaaatcaaaaccgcaataaatccagctctcatcaggacccctccaggatgtccagtgctggagattataataccagtgaacaaaaacaagcctgcaagaagcatgaactctatgtgagtttccgcgatctgggatggcaggattggatcatagcaccagaagggtatgctgcattttattgtgatggagaatgttctttcccacttaatgctcacatgaatgccaccaaccatgctatagtccagactctggtacacctgatgtttcctgaccatgttcccaagccttgttgtgctccaaccaaattgaatgccatctccgttctgtactttgatgacagctccaacgtcattttgaagaaataccgaaatatggtagtgcgttcctgtggctgccactagcgctaaggaattgtggtaataacaaaacctatctgtatcatgtttatgactgcaataaaagcagataaatggggactttcttaaaatcagccctgggtcatttaatctctctatgtgtaatattatgtatatacacattttatttatttaagaatatgtattctcttttggatgccttatcagtaaaatctattttaaaggttcctatattatataatggattcctactctaacttttgatggggtgtgctgagtccagtttaattctatgtcaacattttccccaatactattaaaaataatttctgatattaagtagaattactttgacagtttaaagtttttgaagggaaatgaatatatttggttatatggcaccaatgaaaactaaaatagaatattcagaaatagttttaaaaactagcaacctaaatttaaaagaaaaaacattgcttggctaaatgaaaattttcttttagcacttatgcttcactgtgtaaagtaaaatgttgaattggctttgaataaataaacagtaggcagggcacaggagctgaagggccacaggattgttttagctgttctctttaatgtaattggtgtaaccctaggagcatgctgcttctggcttctggcttgcagttttgtgttattttttgcaaggtcaaagcttcagaaattctcttggcacagacttgagtctatgtccacaagtttccaaatggacactcaaaagcatggtgcaaccttacctcaaatgagtcatacctgaccattgtcttgatcactgatgctgcagtgttttattccccttgtttaaaacttagtccagcatcaagaaatatttgcatgcactccattttcctccctagagacaagagttacttcacgcataaaatgcattggaactgaagatgccagcagcatcgttctcatttacttgcctttagagtagactttgccagttgatgtgatgcaagggattttatttagggtgaagccattgaaattaaactgaagggcaacctaaatttaaaagaaaaaacattgcttggctaaatgaaaattttcttttagcacttatgcttcactgtgtaaagtaaaatgttgaattggctttgaataaataaacagtaggcagggcacaggagctgaagggccacaggattgttttagctgttctctttaatgtaattggtgtaaccctaggagcatgctgcttctggcttctggcttgcagttttgtgttattttttgcaaggtcaaagcttcagaaattctcttggcacagacttgagtctatgtccacaagtttccaaatggacactcaaaagcatggtgcaaccttacctcaaatgagtcatacctgaccattgtcttgatcactgatgctgcagtgttttattccccttgtttaaaacttagtccagcatcaagaaatatttgcatgcactccattttcctccctagagacaagagttacttcacgcataaaatgcattggaactgaagatgccagcagcatcgttctcatttacttgcctttagagtagactttgccagttgatgtgatgcaagggattttatttagggtgaagccattgaaattaaactgaaggggaaaaaaaagtctcagaatggcttagatggagttgccattttcaaacagctgagagctattcttcgtgctttttcgaataagctcagtgcaaacctgattttacaaaaggattttgaaaaccttcctccaaccatgaatgagtacaatagggaagtttctggagcagagtaaaagtgtgtgtgtatgtgtgcgcacatgctcttctctctgatgttaatttattttatctctgttgaaatggttactggtctctaaggtcctttttcaacacacactgactttttgaggagtggggtctttaggttgatcactttctgctgctgttagctgcatgctgaggcttgcaacagtcaactggaaatattcataaggcacgtttcttaatcaccatctgtaactttcaagcacaagtctatcattaggaattttcaaatgtggtggcttgtttgtaattcagatattttacaatgttgttttaaaacacagtttacaatatagaaaatatggtattggctatattggggaatacattaagtaatatatttttagttaataacttaatagtataccattgaatataagactagagattaagaaatagaaatctattatattattctttaaatgtgctacttgaatgtatgtttacagttaaaatatattaaacttgtgatgcca

SEQ ID NO: 79 - DNA, 400 bp, Cricetulus sp.
  source 1..400: mol_type = genomic DNA
agataaaataaatgtagtatgttgacacaatgaacatttattcagccataaataagagctaaacttcatcattttcagttaagtagttggaatttgtgatcagcattttaagagtaataagccagaatgtcagctgctgtttgggaagaggaagaatgcccttggattagaaccagaaaaaaaaatatggaaaagagtaaacaaagtatatgaagagaggaagactcatacaaagacagatgagtcaaggaagaggtctcagagaaaaccaagcctgcaaccattatggtctcaaacctcaaaattctgagaaatatattttttgttgtttaaacaacctagtctctggcatttggtataacatactagccaacttatactgagattccagtgaatccct

SEQ ID NO: 80 - DNA, 400 bp, Cricetulus sp.
  source 1..400: mol_type = genomic DNA
ctcctccctacatgctcccaatttgctcaagagattttatccttttccccttctgtgggggaaaaatgtatgtatctcttagggtcccccttgtttactagcttttatggtggtgtggtttgtaggctggtaattctgtgttgtgtgtctaaaatccatatatgagtaagtacataccatatttgtctttttgtgactgggttacctcattcaggatggtttcttctagttcccttcatttgcctgtgaatttcaaagtcccactttttctgctgcttagtagtattccattgtgtaaatgtatcacattttctttatccatttttcagttgaggggaatccctataagtggaatttctggatcttgaggtagactggttcccattttcctgaggaaaca

SEQ ID NO: 81 - DNA, 400 bp, Cricetulus sp.; identical to SEQ ID NO: 80
  source 1..400: mol_type = genomic DNA

SEQ ID NO: 82 - DNA, 940 bp, Cricetulus sp.
  source 1..940: mol_type = genomic DNA
gttagcgctctacgtgtatgaatatctgctccatgtaggagctcagaaatcagcccaaacatttttatcagaggtatgtcttaagtgttttctgcattacatttgctatataagtgaatgaataatatacacaattatgatttcaagagttgttatatagtatgtgctcgggtatcatgttgtataatctttgtagattttcatgggagcatttgtagtatcacaaaacactttttctgtgcaactcgataaaattctagtaaactttctagccattaattcccttacaaagttctctgagcatggcaataataatcaagaaagtaagtttgctgtaaaagcttctaccaggcccaaggagatggctcagttagtttaatctccagaatgtgcatatgtcaaagactgattgtggtggtgtgcctttccattcccagaaccatgaaggtgaagacaggcaaatagaccaaccagcacagcctgctcttcaatgttaaggccacagagagggtctgtttcaagaacatgtagatgagggccagtgagatggctttttgggtaaaagttcttgtggacatgcctgatcacctgattttgatccccaggacaacattgtggaagaagagaacagactccctctaagttgtccactgagctccatccaaatgccatgcactcataagtctagtacacctgtgcatgcttacatatggacgcaagtgtgtgtatacattaagcttccttcctgcaccatttctaacaatatcttatattagcatttgtttacaagtttggtaatgtggatttgtttgaataagagttggttgttttgaaacgttttttgaaaatcttatttcaagctcttgatattctctctctcttccttcactgttctggtttgattttgttgttggaaacaggctgacctcaaactttctac

SEQ ID NO: 83 - DNA, 525 bp, Cricetulus sp.
  source 1..525: mol_type = genomic DNA
acgtgggggcggtgccggccgaggggtaagttcctcctggcgtgtggtactgctggaggctggagcggcaaccgatcttggatacggcctggcgtccatggaggcatcagcggagcagccaggtccacagctcccgcatgccggggaccactgcatccatgacggcgacttcgtggtgctgaagcgggaagatgtgttcaaagcagtgcaagtgcagcggagaaagtgagtcaagttgctggccctgggcggcgcgcaccttcccctagataagcaccccctccagtgtcccactcttagttctttgaatcgggccccccagccgttgttgccaccactcaggtctccctatcacagagaacttttgcctcttggattctttcgcatccttggctagagggtctcctttgcccttttctcatcctacagcactctcctctgcctttcattcctcaaccttctaccgtcagcgcccgattcccttccaggaattccaggcttggtactccggcccccgaggacgct

SEQ ID NO: 84 - DNA, 400 bp, Cricetulus sp.
  source 1..400: mol_type = genomic DNA
tcactgtgtaacttttaatttaaagttaaatttagggttacatgaaaccagtttgtattagttaaagatattcttagcagtacttgtagtggcatcaccagaaagaatcttctcaactttagaaactaccaagttttacttgtcaagtttactgccttgtttttaagcttgagaatccagattctcaacacactttggtaaaagttacagttaaaatatttttgaggttgttagtattactgtaacttttgtttttttgctcaagaaaaacaacaaggcaacttcttcctgagtgtccagcccctccctgtatccttggcctctattgggcttctgctgtgtctgagctttgaacagttgggagtccagaagggtgataggagggggaataattgagtca

SEQ ID NO: 85 - DNA, 300 bp, Cricetulus sp.
  source 1..300: mol_type = genomic DNA
gtggtgataaagagcttagctttatttcatttagcattaataactgagggaataatgtacataggacacagggccaggcctctcagggaggcagtaatagaaaactgaagataaggtccttccgaatttgcagatttaatagaagaaacggacacctccaggctataatgagccaaagcaggctgagatgagctgagaatagacctcctatataaccctgctgtaccacccctgggaattcaccctagaaactccaagtcagtgtctcacagaatacttgcacaccgatgtttattgcta

SEQ ID NO: 86 - DNA, 110 bp, Cricetulus sp.
  source 1..110: mol_type = genomic DNA
gctgtggactccccaccacgggcaggaggatgggcaggctggggatcctggggaaaatctctgctctcatctgcatcagccacagtaggtaagtgttgggtgttgcacca

SEQ ID NO: 87 - DNA, 143 bp, Cricetulus sp.
  source 1..143: mol_type = genomic DNA
tttttaaaaatcacttgtagttgctgtaatgttcaacttcccggacactgccacagtaaagaaagtggtgaactatctacctcgtgttggtgtgggaaccagttttggattacctcagaccaggtactgttttatctgtaaac

SEQ ID NO: 88 - DNA, 400 bp, Cricetulus sp.
  source 1..400: mol_type = genomic DNA
ggatcccattccttaggtgggataccttgtttagtcttaatacagggggcagggacttggtccttcctgtctcaacttggtataccagacattgttgacttcccaggggaggtcttatcctctctgaggagtatatgggagtgggaagagggaaggtggggggcaggaaagggcgacggtataccaagaactgggcttgatatacaaaatgaaaaaaaaaaacatttaaataaaaaggtgggtgttacttttgcctacctttggggctcttttcctactgttaggttgcctggttcagcctaaatatgaggattttcaccttgccttattgtatcttctttcaatgtgtagactgttgtcacttgcctgactgcacctttttgaacagaaacagaggggt

SEQ ID NO: 89 - DNA, 153 bp, Cricetulus sp.
  source 1..153: mol_type = genomic DNA
tggggattccctatggcttcttgttccaatgttgcctttctgattgggtatactcctatgtgcacatagctaagaacaaatgaacaaatgattgcatcagcatgttaactgttctggattattaaaggaattaaggttgaccttcctgctctg

SEQ ID NO: 90 - DNA, 140 bp, Cricetulus sp.
  source 1..140: mol_type = genomic DNA
gcttatcttgtcagccaagatttattagagtgacttacaggctggggttcagctcgtccaacaatgtctgtctacaattggaaggtccaagaacccagtagttattcagtccacaatgctgaatgtctcagctggtcttg

SEQ ID NO: 91 - DNA, 1138 bp, Cricetulus sp.
  source 1..1138: mol_type = genomic DNA
tttctccctccctcccacaggttatctacctaacagtgcacatgctgtggaggccatgctggccgctgccagtattggagccatctggagttccacctcgccagactttggtgtgaatgtgagttggggtcctgttggagggtaccactagtggggtcagccccttggagcttgtggctgtctgagctgagctgtttgtctccctgggcagccataagagggaggggaaggctacaaggagtctgatctcgtcgtcgttggtgctggatattgagtgattgatcttcacagcctcacacacacccagaatgaacagggatctggttttgactcatttaatttaatttttttttttaaagacaaagtcatactctgtggttcagactacccttgaacttgaggcaatccttctgccttagtgtttcaagttctaggtttacaggcataagccacctcacctgccctggaccttgtttttaagtttactgacaaactggttcttgtttccttcctgcccgctggttcttgcttctctgttagcaggtggtatgtgtggttattaggtggtgtgtgcagtagttattcctggtttggggtcatgatgtcaccttgccttctctgcctgggactttgggactctgaggtttcctgaatttctgtcctgaccttcttgggctttctgagcaaccatggtgctcagggtgtgtccagtgtggtccgggtggctgaagtgtgctatagcaagtgaggaaagcctgctttggcctcagtcattgggtcacttgggccctggactcatggactcacaagctcaggaggtatacttgtaggactcatggactcacaagcacaggaagtgtactgtaggaggacatgcagcactcatgccaccagctctcctggtgccaccagctctcctgacctaggccaggtcagttgatgtggctagcagctgatggttggtctgggtgctgggctggtagagcatgggctgagtttggggtgtgctccctggctcattcttcaggggtgaatgtataaactcacatttactttaaggcatgttctctcgacaaaagtttttgcggtaccgtctcatcttctgtggaggaggagtgctgattgcttagggccccct

SEQ ID NO: 92 - DNA, 1184 bp, Cricetulus sp.
  source 1..1184: mol_type = genomic DNA
ctcactccttcacatccacggaaggataattcccaggattttcactggaggattagtattataaagtagctgtgtcactcgaaaccaaagaagctgaggcatggtgttaactgaggtgaaccagccaccgaggaacaaattccacaagacttgacttacactgtggaatctgagtgtttaggtggcactagcagagtagagtggggtcaccaaaggctttggatggaagggccagccaatttagttacattgatttagctaagccatgcttaatacatacaacgaatgccatcattaaaccatttgagaagcaaaataacaatatgtttttctacaaactaataaaatgaaagattcttgtgagtttccgtagtgtactgtttatcacgctcgcctaacacgcgagaggtccccggttcgaaaccgggcggaaacatgacaagtttcgtccttctcattttctctttctattttcagattcgtagctcattccgtaaacttttcaatttctgggctttgagaatgtttgaccttgacagtcatacttctcaggaccaaagaaacaacagttatttttacgaggatcagttgttgccgaggccacccaaagaagtacaagaaaaccgcaaagatgctgggggattagctcaaatggtagagcgctcgcttagcatgcgagaggtagtgggatcgatgcccacatcctccaacttttatgttgcgttgacacccactcagcagctacagtgatacacaaaagggggaacacatgtctccttacagacttgggtgcctctggaactcaaattccctgtgtacacctttcaaagtctggaaagtagcttagtggtatagtcctgtccaagtgtgtaaagccctaattggattaattaaaatttgagagctatctgaggacacctgctttcttgcattaccttcatcttggttatttgcctttgtaatggaatggggaaattcactaaaatgctcattgtaaatgccacaacacagatctctgccaggttccctgcgactgctcggagggacaaagctcagactgagaaaactgtcaggaaagatctctgccagaacatactttggacctcactgaactgtaggacattggtgatggcttgggcttctgaacccgacattcctcaggtcagtgtctcttgatcaacgga

SEQ ID NO: 93 - DNA, 197 bp, Cricetulus sp.
  source 1..197: mol_type = genomic DNA
tactgtatcctgtcttccagaatcaccgccacccccctattctcggctgtctcctcgtgaccagtacaagccactgggtaagtgtgtcctcctcttgaggggtgggctgaattgggtgctttcaaacagggtgaagttttacaaacactcccacggttgggctgctgcagagagactatttacgtcttcaaattcca

SEQ ID NO: 94 - DNA, 600 bp, Cricetulus sp.
  source 1..600: mol_type = genomic DNA
ttttgtatagtctgctcagtatgcatatacttagtcatttccaaaaatgagtattattattcagcagactttgtaccaaagcatgataaaagaggtggttttttctcagtactgttctgtagccatgtaagctattcacaagtgatttgaaaacatattattttctagaattcatgtgtttacaatgtagacattgttgcctcaatataaactgtacagaagacttttcagagatacttatatagaaaaaaatctgatttttattttgttcaaccgtgtatttacctttgactttgtcctagactgtcttcttgaaaaaagtataatcttgacagcagctgattacttaattgccacttcatttcctgttgcctctctaagtcagatctcagaaagatgtctacacctctaccactggcatcctatttcctgcttcacagtttctatctctggaagtctagcctttgaaggacttcatagaaccgtaactatcaacatgtctatcaccatctatctttctagcttttccatttgttttatggaactaaatatcctttcaaagtatcttccctaattctttgtttctcatcatttcagagc

SEQ ID NO: 95 - DNA, 400 bp, Cricetulus sp.
  source 1..400: mol_type = genomic DNA
ttatcctagaaagacaaaaacaaagccctgtcctaacactacttttgggagttcccttttgacaaattatattggatccaaagaaaggtttttgttagttttataagctagtttagattgaatggctttgctgtttgatgaactatcacctctcctaaacaaaatgtctctcctgtttgaatgtaacctttgtcattccagatggtatatatagtttttattctactgtggaaacaaaggaaaaacctcttccccaacttaaaaaaatatccttgtttccatactgaggtcagtgcatctttaaaatatagaggctgatgcaattcagcagatgtttctacaatccaatgtagaaaaattgctattgttcctttatcattaaatgtaaagtcaataaagc

SEQ ID NO: 96 - DNA, 225 bp, Cricetulus sp.
  source 1..225: mol_type = genomic DNA
tcctgattttatttttgtaggactgaaagacttgcccgagatgtcatgaaagagatgggaggccatcacattgtggccctctgtgtgctgaaggggggctataaattctttgctgacctgctggattacattaaagcactgaatagaaatagtgatagatccattcccatgactgtagattttatcagactgaagagctactgtgtaagtataattcattcataa

SEQ ID NO: 97 - DNA, 25993 bp, Cricetulus sp.
  misc_feature 20478..20489: note = n is a, c, g, t, or absent
  source 1..25993: mol_type = genomic DNA
attagtgatgatgaaccaggctatgacctagatttattttgtattcctaatcactatgtcgaggatttggaaaaggtgtttattcctcatggagtgattatggacaggtaagtaagatcttaattgaaggggctggagagatggctcagcggttaagagcactgactgctcttccagaggttctgagttcaattcccagcaaccacatgatggctcacaaccatccctaatgagatctggtgccctcttctggcctgcagggacacatgcagacagaacagatcttaattgaagttttatgggtttattttagatgtgtctgtgtgtatgtttgtgataggcatgtatttgatacatatgtataatgtatatatttatgtaaaagctagagaaggacattgggtgccatactctgttgctctctgtcttgctcccttgagttggtgtctctcactgaacctggggacatgctgattcggttaggctctgtcccctattgctgaagtttcaagcagatgcggccacacctggtttttatatgggtattaaggatctgaatttaggtcctgatgcttgcacaagtgctcccaatggcctgcatgtatgtgcagcatctgcattccaggagaccacaaaagtgtgatgggtcctctggaactggagttgcaggccattgggagctgctgtgtggctgggaactggacttagggcctctgcccaaacagcaaatgggcttaactctctccagccccaatattttctagttttaaagttctttttcacaaaaagaaaaacaccaggaagtcctactagttattcagtttgagatataaatagtggtactccataatgagtccagcaaatgttgaaacgaggttggaagtctgtaaatgagattatgattctgttggtttgctatacacatttcttttaaaatttttttataaattttatttaaattagaaacaatcttattttacatatcaatcccagttcactctcactcccatcctcccaagccccccaccaattctcccatccactccccaaggagggcgaggccttccatgagggatcatcaaaaaatctgtcacttggggcagggcctaggccctcctcccctgttatacacatttctttactgtatcactgtatgtaaagtatatatacacatacgagttatgtactatcatataatactaaatttttggactttttaatattagtacatctatagctatcacatgtttttatctttaaaaatttttataattgtgtgtgtgtgtgtgtgtgtgtgtgtgtgtgtgtgtgtgtgtgtgtgtggagagatatgtgtatgtgtgtaaactgttttctctttgagataggttcttactgtttagttcagtctggcttggaactttctgtatagcccagactgaccttaactcagtattcctcccccatctggagattacaggtatgagccaccatgcccagtcctgtttttatattgattttgaaaaagggtctcaaatagtttagactggcttccaactcacagtgtagctgaggatgacctgaagttctgatgctattgcttctactggtgtgccattatgcccagtttgcacagttatgaggataaaacctaggaccttgttcctgtgcatactaggcatagactctaccaattaagctacattcccagctcccccaggtttactttaaaggtgataatgctattctgttcatggatataccataactgatttaactctgtactgttggtagacacttaagatctccattattttgcacttagcaggagtttattagaggaaatatgatatatattggcacttgctcagctttaggcaagtagaaattctgggtcaagtaaaagatgtttattgaatattatcatactgccaaattaacctctaaaaaatttcataattttatactaccagtggtatgtgagttcatttttcctcacaatctcttctaatattttacattagagaaatgcaatttaaccaaaattcaccagtcttgttagctttgaacattgtatctttttatttgtatttttaaatttcatgatgttatagtatacctgttcttttatttgagacagaatcttattatgtagccctaagtgacctagtacttactctgctgttgtacagtatgtccctcaacccatacccttcctcctgcctcagcctccagagtgctagaattataggcatgtaccatggtgcctagcatgtacctggtttttaaaggtagctggaattataggcatgtaccatgatgcctagcatgtacctggtttttaaagttagtaagttttaattttggttgagctgtgtgttggtgtgtaagtataatctcagcacacagagttcgagacatgggttatgagttactatgcagcctgggctacatagagggatcctgtgtcaccttccccaacccccaaaatcttccctttgccatatggaaaacatcccccactttatttaatagtttgatttatgaagcaagattaccaattatggggacaaagaatgtgtcctgtggaagtttaagaagtgtttgttataaaaatataactatttggaatcttctattcctgattttatttttgtaggactgaaagacttgcccgagatgtcatgaaagagatgggaggccatcacattgtggccctctgtgtgctgaaggggggctataaattctttgctgacctgctggattacattaaagcactgaatagaaatagtgatagatccattcccatgactgtagattttatcagactgaagagctactgtgtaagtataattcattcataatttaaaaaatatggcaatcctagttttgtatgtatttttgtttgtttgtttttactttgaaacagtgtttttctgggtagctttggagcctgtcttggaactctctgctgtagaccagactgacctacagagatctacctgcctctgactcctgagtgctgagattaaacgagtgcaccaccacctgagcctcatcaactgaggcttatcccagttttttttagaattttagtttgagaccctctctaacttgctcagactatccttgaactcattacataggctgatctagaaattttagtcatcttgactcagtttctcaagtagctgggattacaggcaggtgctaccacttctggccataattgttgctgttattattcactggtactttacatcacaacactgtagttcaagaccaacatcaaagtgaatattgtttgttagtcacccaagatggatttgtacttactttgcatggtttttcttttgtcttattttgacctggattttgacttgttttatgatactgctcaggctagtatcaaactctgtggttcaaataatccttctgtcttagcctcccaaattctgggagtacaagcgtgtatcaccatacctgactgtgttttgatattttccaaaacaacttttaagaattactgcagagtacagtgacagagagagatggttcatcagtcacaagcacttgtagagaagactggggtttagttactggcacccaaaagatggctcacagctatgtagctccagttccaggggacctgatgtcctagcctccagataaaataaaaataagtaagttttacaagaattatgggactggtgaggtggactcagtagtcaaaggcactggctgctctttcagaggacctggttcaattcctagcacccacgtggtagttcacagttgtctttaactccagtcccagggatctgatgccatcttctggcctccaagggcaccagacactaacatggtacacaaacaaatatgcaacttgaacacccatacacatattttaaaaataaacaatttctgttgagacttggtgttttcctgactgtatatgctggtggcaaataatttctctggaactaagttgctttgagagtaatcctaaattatttgaagcaaagtctcattattaaggttaacaattatttctcagaaaacagaaaccacttttgacctttggccacgttacctttgtcagagtctgaagtgatgtagaatattgacaatatgatgaaacaaaatgaatgtaatctttacatgtaaattccatcattcacaattttgattgtgtggcttattttttttattttcattctctctctttttttttttgagactctatgtagctcttactgtcttggaactcactgtatagaccaggctggtcttgaactcagagacccaactgcctctacctcctgagtgctgggattagaggcatgtgccaccactgcctggctaaaatagatatttatttttaaaataaaactgcaggatattatagagaaacttaagtcacccatattctttttgttttttgtttttttttttgtttttggtttttccagacagggtttctctggctttggaggctgtcctggaactagctcttgtagaccaggctggtctcgaactcacagcctgcctctgcctcccgagtgctgggattaaaggcgtgggccaccaacgcctggctctctttttggttttttaagacaggatttctctatgtagctttggagcctatcctggcactcgatctggagaccagactggcctcaaactcacagatatctgcctgctgagtactgggattaaaggcgtgtgccaccaacgcctggctaagtcacccatattctatttttttaagttttatttttattttatatttatgagtgttttgcctacatgcatgtgtgtttaccatatgagtgcctcatgtctacaggtgccagcagaagacatcagatcccctgggactggagtttcagatgtttgtgagtcaacatgtaggtgctaggaactgaacttgggtactctgcaagaatagcacatacatttaactattgaaccatctctccagctcttgtcaccactttttgagtattttaaagtgttttctgtctgtatacatgtacacaatgctactatagtttatataatcagtgttatcccagatgtcatttaactaggtggaaaagaacctgggggttaccaaatatgtatttcattaaatgaaagagtctgctaattaaaacctctgtattttttgtgcttctaagaaagaaaatacctccagttatagtagcatggtaccatgaattatgttttcagaggcagtaaagtatgaatatatatcttaaaacagtgaaatatagtgttatatttggtgacattttttggtcccctatgctctataaccttcaggaaaatgaaacatttttacaagtatacaaatgtgtttcttctattttgagttttatttcaattttataaaattgcactaatagttgatgaaaataatgaggttttgggagaaggggtgttagcaaaccattattttaagttacatttttagatttgtaattaatttattttcagtgttgaatgaatccaagacttcaagtatgctaagcaattacgttgctacttagggattttcaaagatcttagggatggaaaattaggaataaacaggtagacaacgtagaaataaatgttaaaggctgtattttagacccttgagattctctgggaattaagagatttttatgtgtatgagtgttttgcctgcctgaagtctacagaggtcagaagagtgtctgactttctagaattggaattgcatacaattgagacctgccatgggtgtgcttggaattgaacctgggtggtttggaagccaatgctctgaaccacctctgggaatttttggttactgacagtgacacctaaatgaaggaacccttgggcactctgcttaatgaaatgatgaaaagggaaagaagagatggagccaatctggagagaactgtcctttcattcgttccagtcatgtctccatagagtttattctaatagttaaaggtgaggcctctctgagagaggcactaacttaaggatacagtcagttccagatacagggaggttgctgtggtttttctgcccactcaagctaggtaattgattcatggatagcatttgtaaacaatataggcatttatatataacaactattaaatgaacattttattagccccattcaaaaggaggctgttttccttggaaaaagtttcaataaaacataagctgaaacagtgaggtcagtgggactgtggaaatttgaacagcctttttttggtttttcgagacagggtttctctgtagctttggaggctgtcctggaactagctcttgtagaacaggctggtcttgaactcacagagatccacctgcctctgcctcccgagtgctggcattaaaggcgtgcgccaccacagcccggccttgaacagtcttaagagtgccaatgttctgtgtgctttggagttctgttgcctcggaccctatagccatagcctttggatcacagcttcattcttaggtatttactccttttgtcctttccttgaaaagggaattaattggtgttggactgtaataccatgtttctacacaagatgtcagtatttacctaggtttgttattactatgtagcactttagctgtaaactacagctgaaaacctataaactagtttcaggttttctacaattatttttctttttgtcattaattagttctgtgtgtttgagattctgggttttagattagagtaataatatgcaaaattacattaaaaagccaaacatttagtttacagtatgtactagtagtatgcttttatttattttgttttgtttctttcttcccacttccccttacacataccagacaaacattctaccaattgaattatcatttgttttgttttgttgaagtaagagtttctgtgtgtaatcttggctgtcctggaactggtgctatagaccaggttggcctagaactcacagagatccacctgcctctgattccccagggctgggattaaaggcatgagccaccaccacccagctatcttttttaatgatattttgagataggatcttgttaggtagcccagagtgatctcaaactctcaaccctgccttagcctccatcctggaacacgctctgtatgccatgctaggcttaaactcacagaggtctgctgtcctctgcctcccaagagtgttgagactaaagatgtgcactaccaccaccaccacctggcccaacatacttttgttgtgttttgtttttcaagacagggtttctctgagtgacagcccttgccatcctggatccacctttttctgcctccctagtgctgggattaatggcttgtaccaccaccatctagcctaacatacttttaaatacagaacaaaaatagttttataggtagcatatttgtctgctacttggtattattagttaatattgatccatataagtatacattaattttttgtgattctttttaatagttccatagtattattgttgtgtacattaacttgatttatttagttaatcccctgttaccacatgttgcctctttttgatgaaaaaagaatacagtgggctgggcgttggtggtgcacgcctttaatcccagcactagggaggcagaggccagcctggtctccagaatgagtgccaggataggctccaaagctacacatagaaaccgtgtctcgaaaaaccaaaaaaaaaaaaaaaaaaatacagtgggtactttatgttttttgcatgtttctgaatgtttttgtgacattgattcttggaaatggggttgttagaccaaaatggtgtgcatgtttccagttttagtacacattgctgtattcttttcaaaagaggtcatattggtttctgtactccaaccaacaacttgagtatttgtttcccttcaatgtattactaggatttagttctgaaatgtggatgggtgaacactggaatcacatttttttagtagtgcctttttttgttttgtttttcaagacagcatctctctgtgtaaaagagctctggctgtcctagaacttgctctctgtagaccagactggcctgaactcacatagatccacctgcctctgcctttcgagtgctgggattaaaagctgagctgccaccattaggctttagtagttctattttaaaaagatatatttatcttactttatgtacatgtgtgaatgccagtatgcataccatgtgcatgctcgatgcttttgaggctagaagggggattagaacacttgaaactggagttacaagcaattataagctgccatgtggatgctgggaactgaacccaggtccattgcaagatcagcatgtgttcttaactgatgaactatctctccagtcccatctagtgggttcttattttgaagtataacatgcattaagaatattggttatagatattttcagttggggttcatgacctgttttttgccctttctagtaatgcaaaaaaaaaaaaaacaaaaacaaaaacaaaaagtatcttactacatttacttccaattcacttatagttttattttttgtatttagattttaatccatctggaatttatttttgtgcatggtgtgaggtagggaccatgctttatattttttctcaaatggataagtaattgaccagatgccatttgttgaataattcatcttttaattactgactcaagatgccatctttgtcatatactaaattgccatatgtttttgggtctgtttctggattttactttgttcctttaactttttgtcaatcactgtactgattgacacaagggaattactgtagctttatggtacttgtcattttcctagtagagaaagtccaggctttttatttatctgctttcaattcagttttctgtcttcatttcagcacatccaaataacagttattcccaccaactacagtgagtgttaagtgctctcttttgaaacccctataagggtttctgctaatttctatgagagcagagtgaaaacctaagaagtgtgttctaacagactggtactcagacctggtttctcagttctgaagatgacttgggtatgggctggactttgagggggcatatcctcagggttggatgtgatacataccagctccttcaacttgtgactcacaggaatgttacagggccagaagcagacacctgccatgctaataaattgtcctttgtggttgagaactgcctgattgatgatgagcagctatattatgctcactcagaaatggatgaactctatttttaggactcttcagaacattaaaaaccagcaatggccagttatgatttttaaattttactaatctctatttacctagtcattaatttctagaaacctaatacttataatacatgatttagaataccataatgttgtagccattagatagctaacttcttacatctaataattttttgtgtgtattcaagaatatgcatgtaaatgatgtttttttttaactagaatgatcagtcaacaggggacataaaagttattggtggggatgatctctcaactttaactggaaaggtaagtaccttaaagggaaagcaaagaatcatttttataccaagaatcaattagtaactgtgctttcaatcactcacttggagatactttaagtaagcaattattgtttaatacttgcaaagtacccttatatagttttatatatcttctggactatatagccaaatgacagcagaatctaatagtaacactaaaaatccacattgttaattactgcgttctgtaatacctctttgaaaatctaatttggtatccttcagaaagtacaaatggggagaaaggcagtcttttaaaaagttttatgccgggaggtggtggcacatgcctttaatcccagcactctgcccaggcagaagcagcctggtctacagagtaagttctaggacagccagggctacacagagaaaccctgtctcaaaaaaaaagttggggggggagtttcacatgtagttttatttttttaaagattttatttatttattatgtatacaacattctgcttccatgtatatctgcacaccagaagagggcaccagatctcttaacggatggttgtgagccaccatgtggttgctgggaattgaatcaggacctctggaagagcagtcggtgctcttaacctctgagccatctctccagcctcggctttcaaaatcttaaactgaacattgctctggttttgtttttgtttaattttatgtgcattagtattttgcctgcttgaatgtctatatgaaggtgtttttgtttgtttgtttgtatttgagacagggtttctctgtggctttggaatgacgtgcatgagtttgttgagttcaggagccagttctttccagcatgtagaattgagagttgggagtatgttaacatgatcaaaattggcaccaagtacctttactccctcagctatctcactttgtagcccatatcatcccaacatctcctcctgcctcagcctcccaagtgctgggattgcaagcatgagccactatactggactgaatatgttatactaagctgctagttatttacttcagatactgtgccttgtataaatgtattactttggtaaatttcacagtaatctggtaagggaagttatattctcattctgcagatcaggaaattaagcaatatagttacagtttggaaaaatacatgggattatgagacaactgtctcacaatctcaaagtcacacagccaggaagtagaagagcttgaatcctgacatcagagttctttcttggtctagtttttacattttagtgctcacagtttacttcgttgaaaatctgtaggggaactttaacataaaacacttgtgaaattgtatagtcctgtatgcggtcatggcaaactaatgttgtcatcagcggtgtttggaaacttcacacaacttgtaatctgttggttcattctctctcctgtccatttggtatacaactgccagacctccttccctttcattgtgccctgtccatttttagaagccattcaaaacctgcacttgtataatcagtttttagtgctagaaggggctttgccctcagtgccttctgtgatttagtttgatcttgtctttttcacctaatatttctgtcccactacacagctctgcgatcttaaacagatctctgcttctaaagtaactttatgacagttgcagtagattctgtttattgtggataaaatcaaggaataaaaattaagcggagttaaatcttagtggtgagattatgggtttattgaaacatttttcttttgtggaaatttatcttatgtgtgtacacatacagacatacacaaatattttcccattaatacttgagaatgtaggctttgtatctaaaatctacttttattcaaattattcctcttgtacttgtctttgttgattacttaagtcatttgactccttagccttattgttatcattattaaatagacatgtagcaacttgaaataaaactctcagtggtagcaattcttagttttggtaatggagcaatagcctagtacagttttgtattcattggacaaattttaatctttctggactttgttgatttatataagctgtcttatgctaaaatacatagttgtggtccagcaagatgtcttgatgcaggaagacacttgccactaagcctgatgacctgagttcaatacctctgacctccatattcaagctgtgttacaacctcccaaatgaaacataaagtaaatgtaaagaattaaaagatatacttatgactcaagcaaggtcattagaattcagtagttctgcctgtgtcctagaagcccatgttgtttcctagtctataattctgtaatgctacagcagcagtcactgccgctgtgaaagtgctttttgatgggactgtttgctatccttgaaatgttcaaggtagtggtgagaaaatggtgagaaaactgcaaagtaggtagggaaacaattactgactcatgagttaactatatattcagtttggccaaagatttgcatagtagtgtcatgcaatcttcattcttgccctgtaagggaatcatcatcctctttctatagagaaaggtattgaaatttagtcattatcaggtgacagaaccacaattggaataaataaatgccttagtccaatgtttcttacactgtgttaagatttatttcaatagctccattgctttttttctcagtatatgaatgtattttcagtttacttagaatttatatagacctcataataatatcacagttatgttattcatttgctctgtaacctgtacctttagattaccccaattttttatatttgttttatgttctatgttatcctgagctttaaagtgaccttttcttgtccatagaatgggctgcagttgtgagttgctttccagaagttctgggctaaccttgcttgaatattctattcttagacccatgcaggactgtcagatgctgccttctatataataaagtgctttgagacatgggcttattagtttttcttggttacagtttacttttaatgcataagctgtgattctccaatctttgattttcatgcccagtcactaaacagttttttaaggatttacccatagcatatgcatttgtaagttacgtatgaattttgctagtaatttatgttgtaaagtacttaaaactgtaggttaaactagaggaaatatgtcttcagttatttttctattgctttgaagacacaccgtgacccagacaacttataaaaaagaaagcatttaattcagagcttgtttacagtttcagagggtgagtctaggaccatcctggaggggaaccaggaggcagactggcagcatggcattggagcagtggccgagaacatatgttttatctgcaaattgggtgcagaaagtactagagactggggctgatgtggacttctgagacctcagggcctactcccagtgacaaacctcctccaacaaggccacattgactaatccttcccaaagcagttccaccagctagggaccaaacattcaaatgtaagagcctctgggggagggacattctcattcaaaccaccacaagatgaaaaagttgaatttcatgaaacccagtggatgctatatgtactctaccttgataactactaacttagaataatatagagggtgttataactctgatgttctggttgtttggcagagtgctttgagaactatataaacagtatagaataaatcttctgtgcagtgagtattgtgaacatgtaaaacgctgctttgtttctttaactagcataaacacaggaaggttgagtgaggattccggcatggtaaatcagaactataatcttagcatttggtagattgagacaggaatattgctgctagtttgaggccagctttggctatataatgagttctaggccagcctgggctacagaataaaaagagaatagattggaatgggtttaagggtttgtcgtaaaaggaaggattagtatttttccttagtgtagtcccccagatatgtgtacaaccattacactagctttagttgatgggaaaataaaattttaattgcatctctgttatgttaaatacatgatttttggaggcatttgtacagattacacttagaatattagagtcaagatacatgaaaggccagctggttcaggtttctcagtttacatatgggacacagatccagagaaatttcacatgttgcttaagatcttgcattcaggattaaaagcttataaaaaatgaacatatgggtcaaatattctttctaatagtatacttgtttcaagcaggatacagtataatcaaatacaagcaagaatttcccagagttatatgattttactgataattggaaatatatctcacttcattttctgattttcatttctcttttttcttctagaatgtcttgattgttgaggtaagtgctgcatttatttctaaaatactgttaaattttctagcttagtgtatactataggtaagccagggaaaaattctttctgttttaattggggggggggtgttttctaaagactaagagaagccatgtacagtaaggcatgcctggaatcccaaacactcaggaagctcttgaattccatttaggttagcctgggttacataatagagttcggaaactagagagttcttgtttttaaaataaaacaaaataaaaaaaacacaaacagaaaaggataagtgatgtattttccagctcatgaaaaaagtttctcttattgttttgattggtttcttttctttcttggggggatggggggctcgaggcagggtttctttgtggctttggagactgtcctggaactagctcttgtagaccaggctggtctacaagaacagagatctgcctgcctctgcctcccaagtgctgggattaaaggcctatgccaccaatgcccagctggttttttcttttaaaagtagctataactatatagaggggtatgtgtgtgtatgtgatttactgtgctaagttttattttatggaacatcataaggttcttatttcatcttaatatgagtatcaaatattcttagaaaaactatttatagttcatgtgttattttccatttgctattatgtctttgagtactttgtatgtaataccagtaaagtttatccagtgcttactttgccagctactactgtaaatgatgtagaaacaagcacaagaagggtaaacagattcccaaggtcaccagagagtgagtagagtaaagattcaacctatcactgtctgtagcctgttgcttttcaatgtgatccctaaaaaccaaaagcatcatccaagcaactactgactcacaatctttatgatgaaaaccagtgagcttttcttaagcaaactttgagctaagctttaagaaccaatgatttacttagaaactttctcattaaggccttctttgactgtgctacataaaaatcttgtatttccatgtcaacttcctatccctttcagtattttttcacttgccactgtctaacagcatatacatatacctctttaatagtctatggtctccctatttcatatctgtctgccttactgaactctatacacctgtcagtgatcatcctcctaccttgacgctgctcaattcttcagtaaacattttaatatatttttagtgtggcttcaagaacctcattctccattatttttcattttctctatactttccacattttccagaacatcgtttttctatcatttttctgcccctagttgcttactcgattttataatctctttgtttcctttatgcttttttgtaatatttttgtgcatgtgttttgtatgtctgagtgtatatgcatgtgtatgcaagtatgtatgcctttgcataactacagaggccagaagaggatgtgtgtcctttattgtgctgtctattcatttgagacagggtcctctctctgaaccaggggctcattttattagaagccagcaaaccccagcagtcttcctgcttcttgccttctcagacatgggctgcaggtgggaacaagatgcctagttttttgtttgtttttttgatttttcgagacagggtttctctctggctttgaaactagctcttgtagacctggtctcaaggctggtctcaaactcacagagatccacttgcatgtgctgggagtaaaggcatgtgcccaccactgcccatcagatgcctagtttttatgtgagtactgagatccaaactccagtgctcctggttcccattgagcatcctctgcagtccttattttgtttttttttaacaaaagaaattttattttttaagtggggctctaggaatcaaacccagaagatgctaaataaatgttctaccactaggcttcaccctcagttctttgttttgttgtttgtttgagaaaaaggtagctaagctaaatagtgcaggatatattcaactccatccacctgcctctacctcccaagtcaagtgctggcattacaggcatgtaccatcatctacctcccttctgcccttctctttctcattccatctctgctctcctccctctcctctttctctctcttgcctcctttcctctcacccttccttcagcattatcttggtttttgtttttttttaaagatttattttatttttattatgtatacagtgttctgtctgcatgttgacttgcacctcagaagagggaatcagatcccattgcagatggttgtgagccatcatgtggttgctgggaactgaattcgggaactctagaagagtagccagtgctcttaacctctgagccatctctccagcccccctagaaggaacttgttgtgcagctcaagatggcttttttcttctatgagacgaggtgtgtagccccatttggtctgagtctcacagtgtggaccaggatgacctcctctgccttcagaatgctaccattacagttgggtatcaccgtacctggcctccaagctggctttgaacccactgtgtagcttatgctgactttgaaccccttctgccttagcttctcacatgatggaattacaggtgtgcaccaccattgttccattagtatttttttaattttttctttttcccattttagttctttgagaatttggtatattgcattttgattatgttcacctccactccactccccccaaatctgctcccacttccctatttacccaacattgtatcttcttcttttctttaaatccaacaatacagttagtgctgtacattattcctggatgtgtggccttcactagagagtggtcaccccactaggagccacattgttaaagaaaactgactcctctttgcaacacctaccaattgctatttgcaattaaaggtggtggtagtggcacacgcctttaaacccagcactcgggaggcaggggcaggtagatttctgtgagtttgaggtcagcctggtctacagagcgagttccaggacagcctccaaagccacagagaaaccctgtcccaaaaagccaaaagaaattgatccttcactagaggtaggacatgatgccctcttccccctccatgctggaattttgtctggcctggccctaaacaggttttgtaatgctttctcaactgatatgaactcataagtgcaactgcagtactgtgtcagaaaacactgtttccttgaagttatctacgactttgtggctctttttctttaattaaaaaagtttgttgccaggtgatggtggtacacacctttaatcccagcactagggaggcagacacagaggcagaggcaggcagatatctgtgactacagacagagttccaggacagaaaaacccacacagagaaactctatcttgaaaaaaaaaatagtttgtatttgtttgcatgtgtaaacatgccacagtatgcacatggaggccagaagacaactttgaaattgcttcttctaccttttttaaaaagtttaaattagaaacaagtttctttacatgtcaatcctatgctggtttcccagctatcagtctggggtccataagcaaccccttctaccttttgcatggaatttggggattgaatttaggtcactgggcttatatggagaatggctttattcaccaatccatcttggttgcccttaattgttatttttaagagatttatttatttataatgtacaatgttctgcctacatgtctccctgccggccacaagagggcaccagatctcattatagatggtggtgagccatcatgtggttactgggtatgaactcaggatctcaggaagagtagccagtgctcttaacctctgagccatctctccaaccaccttacttttttcagtaatattataaaaacatcttcatctcgctacgcagaaatcagtccccttagcctttatttatttatttatttatttatttgtttgtttattttttggtttttcgagacagggtttctctgtggctttggaggctgtcctagaactagctcttgtagaccaggctggtctcaaactcagagatttgactgcctctgcctctcgagtgctgggtttaaaggtgtacactgccaccacccggctagccttcattttttatttcttccttccccttctctactttctaagatttgcctattgaggacatagcacaaatggaattacatagtaattaattgtactttgtgtctggcttttttcaattattttaagatagggtctcaccatgtggccctggctgattagctatgtaatcgaggctggctttgaaccacacaccccacaaataaataaacattcactcaggatgatagcactgtatagttacagtaattatccaaatgaggcatagttttctctctggctgttggccagggattgctctcatttacagagatcatccctcatgtcctacacagcctctcatagcatgcctgctcacttcttccactctgtactctagggggcggtaatttttgtgccatcagtattctgcctgccacaccctagaatcttggtttttcacaagtctctagccctgaatttctaatgtccacattgtcttacgtatttttttttcttttggttttttgagaaagggtttctctgtgtagcttttggagcctatcctggcactctctggagaccaggctggcctcgcactcatagagacccgcctgcctctgcctcccgagtgctgggactaaaggcatgcaccaccaacgcctggctttgacttacggatttttatttgttttctttttcttttctttcttgttgtttgttttttgtttgtttttgtttttgagacaggatttctctgtattattttggaacctgtcctggaactcactctgtataccaggctggcctcgaactcacagagatccacctgcctctgcttcccgagtgctgggattaaaggtgtgcactaccaacgcccgactttttatttgttttctgataccagttttggtaatactggagatctgcggctcagtggatcttatgcaggtcttttacccctgataaagtcattttatatagtgaaacttagccatttgaatgccttcaaaaataatacaaagcaccatttctcacagtattagagtttgagatgaaagagtccctatcttgaagttatggagaaaaatctaaaatacttttacagaattaagcatagtggcttatgtctgccatcctagctgtcaggagtctaaataggaggctcttgagtttaaggacactagcctgagctatttagctagacccggtgtggaaaaacaacaagaagatttacatgtatgtacattgcttacatgtacccatttcaattcattgtttttgacctcactatacctatgcagcctagttctgggaactgacattcccactacttgctgaaagccagatgggttgcaagttctagaatctctgtgtgtgaattgtgagaatttttttatgtgctgaaaaataatgacatcgtactgtggaataccagaacactgggattctgtatattctgtgtttagatagtgactctgattttaaaattatgctggttttacttaccacttaccattaaatacctcttttctttttaaaaggacataattgacactggtaaaacaatgcaaactctgctttccctggtcaagcggtacaaccccaaaatggttaaggttgcaaggtatgtatgccattttaaagtagaatgtttttttttcattgggagcaataagcaattgcttctttgtaaggagaaatgctttcagctctctttttatcttgaaaaggtgatgtaatcttaaaatttagtatactttgttattaaaacccttcattatatagtcaactacttgtctaatacagtatatacagggacttggattcagaaccacctcagacactaagctccctagatgttcaagtcctttgtaaaaaaaaaaaaagtcagatgggctggcaagaggactcagtgggtgaagtcacctgctgccaaccctgatgacctgagttcaaaccctgggacccacatggtagaaggagagaacggactcctagaaatttccctgtgacgtgtgtatcacaacacatgtcccccaacccacagacaagtaaatgtaatttaatttttttaaatgtcaagagtgttttcacagaacctatgcatattctcccttatactttaatacctattgcaatgtcatataaataattatgctgaatcatctaaggaaatatgtatagttttagtatagtttcttagactatttgtgtgtgtgtgtgtgtgtgtgtgtgtgtgtgtgtgtgtacatgcaggtgaatgcacacaggcacatatgcaaaggccagagcaggattggatatcttcctctattgtgtaatcagggtctctaactgaggnnnnnnnnnnnngcaggagtttggtgggtgagcttttgcatctctctccaccctcacagtactggagtcacaaacacatacagccatggccagatttttatgtgaatactgaagaggcaaactcaggtacctgtgctttaggaccaacttgatacacaaggtttagatacagaactctcacacatatgtagagtcaagtaacaattggttttgccacttgctatttatatcagagttgtcctgagtagcatgcaaggatggatctaggagcccccttgtgaaacagaaaccggcgatacccatagggttgggctctgaacatgtggatctcctgaagttgagtggtggataaggcatcctgttttgtgtcatcattgacattttacaatacctgctttttttctgaagcagtcacatatcttcctggccgtatctagatgtttggctttgctttcatagttctttctgtttttatccttagttacatagtttgatcctacaacagttgttatcgccatcttacagatgagaatgttgaagcgtagggaattgcccacagacagagctaataagctgtagagagatgatttgaatacagggtacctggctcagttgaggcttctgttattttaagttggggtttttgggttttttgcttttgtttttcgagacagtgtttctctgtggctttatcaggctggtctcgaactcacagagatccgcctgcctctctgcctcctaagtgctgggattaaaggtgtgctacaccaatgcccagtttaagttgtttttttttttattgttagaagtaacattgttttgttgttgttgaattttgctttgtttttactttacaatttcatttttattttcaaatctttgactaatctgtgttttttgtgtagaggaaagttcgggtctgtttttcttttttccctgaatgattaactggttcttgacattttttgaattacacttatttattgtggcagggatgtgggcatgtgccacatgcacttgtgtaggtcagaggatagcctgggggagttaggtctctaacttgttggtcgtaggtgggggtagaatacagcttggtagacttggcagcaatttccttacccactgaaccatctctgctgaccaattagctgccttttataaaccattttaaaggatcaaagtatcttgtaaagtctttgaatatcattttaataaagcctatttgagcagttcatcctcaaggactcttctgatagatggtgcaagtgtacctcagtgagtttttgtcttgctggaggtgctatatgctcaagagaatgggtaatttttgataatgtagttgtcctatacaggtgtgttttgtgtttttcccaaaataaaaattgaaaagctattatttgatatgaatatatgatagtatggccactctagaaaacaatttttttttgaaaactgtacatgtaactaccagaaggccttacaaattatactgggtacttatacctgagaagtaaaagtgcacattgatataaaaatttatataagttgatagctaggtatggtggttcacccgtgacgtcagcatttggaaagtggaggcaggaggtcacattgaatctaagtcaagcagcctgagttttcatagctgtctcgtagcttcctactcaaatctgtaccagagtattcacaacaccatttgtgatagcccacaattaaaagcgacaaactgaacaaaactgtagtacacctataccatcatattcatcagtaaacaggaacaactattgattgatatacaacagttcagagaagtctaagtggaactgtaccaattatgactttcagtagtacataaagcaggtgttttgctctgtttaaactgcttacctgccccattttcccccaagactattataagcccccctcaaagaaaagtaataaaactgtatgattctacttaaattacctactttgaaatggcaaagtcatacagatgagaacagattaggggttgccaggggttaagggaatgcagaagggagccttgcatgtttcaaatatcttgattatggtagtgattagataattccacatgtgactaaaattgcctgaaccgcacaaaaagagatgagtttatataaaattggtgaaatctgtacatggtcacttagttcatcaatgcccatgtcctggtttgataatatgctgaaaaaggacactggagacctcccttttgtatgatttttttcatttgcaacttcctgtgaaaataagaaacattgaaagccattaactattttggtggtattagggattcaactcagggttgtatgcatacatgacaagcattcagataattagctttagagctgtatgtaattaattctgtaatcaaattacagttgcaaattcatgatttctataagtactcaataacagcaaagtaattttaaaaaatgaaactgctcaaagctattatgtttttagcaagagtcaaacagttgaaaatataattaactccaaatgatgtgagggacgttgatggtcccccgtggagggcttcactatccatggatgggggtgggttgggggaggtcggcagagagcataggaggatgggagggtgagggaatgggggactgatatgtaaataagagtgcttctaaaattaaaaaaggaaaatataattaactaaaaaatggagtaggtagctcagtggtaaaatacatactttttttgctggggggagggggggatgagttcaagatggtgtttctctgtggcttttgtggctggcctgctctgcggttaagagcactgactgttcttccagaggtcctgagttcaattcccagcaaccacatagtggctcacaaccacgttgaatgagatctagtgccctctgctggcatgcaggcagaagactgtatacaaaataaataaataaataaataaataaataaataaataaataaataaataaataaataaataaataaataaataaataaataaataagtaaataaataagtaagtaaataaataagaattattttaaaaaatggttcttttttttgagacagggtttctctgtagcgttggagcctatcctggcactccctctggagaccaggctggcctcgaactcagagatccgcctgcctctgcctcccgagtgctgggattaaaggcatgcgccaccaacttttaacacacttgttttgtagcctaaagttcccatttgtgaggcagttattaagagtcttgatgaaagagcattttttacccacctcctttataagttgaggggttgtatttcaaggattctgaatctagatcccttatttttgaatctttgtgtgttggtaccttggcccattcattattctcagtagctgcacttcaaaggacatatgggtagttaggattccactctcagaagaagcatcttatcaggttccaggtggagatcagtcaggggccttgtcttgttgtaaactctgatgtgggtctccatttgtcttcctatgggtatgcaaaactgaccttattctaaatgcagatgatactatttaagaacactgatgatgttttcttttataatgttctattgtctttccatatgtcacatgtaatattttgtaattaacagcttgctggtgaaaaggacctctcgaagtgttggatataggccagactgtaagtagatgccctttgggtgatcattttatctatcatagctgaaaggtgactaaaattgcttagagttattttagagaacgtttgtaaagaaggcacatttgcaaatattagatttgaaagtactgttttctctgtctaaataataaactgtgattctttacagttgttggatttgaaattccagacaagtttgttgttggatatgcccttgactataatgagtacttcagggatttgaatgtaagtaatgtttctttttttctcgctctcatttttcaaaaaacccacataaaaatttaggaaaggaaagaattgttttctccttctagcacctcgtcatttgaccagactgatggttcccattagtcacataaagctgtagtcaagtacagacgtccttagaactggaacttggccaggctagggtgacacttcttgctggctgaaatagttgaacagccttaatttacaataactgcctcattgttatttcacatgataaatatagtcataaataagaaataaataattccatttaaatcttatgattgaccctacttcagatacctgcctcccactctggaggcaagaatccctcaggtgagtttatgaacatggtccaggagaaattaagaagactacgtggacagcattatctaaagcagttttgtttgtttgattggttttcaagatagagtttctctgtttaaccactctggttgtcctggaactcaatttgtagaccagaccacagagatctgcctgcctctgcctctcaagtgctgggattaaaggcccaaagtacagtttttatttcatttttttaatcacttaccctattttctttgagacaggatttatcttgtagtactggctgtcctgaaactcactctgtagaccagattggcctcgaactcacagagaatccatctctctctctattgggattaaaggcttgtgccatcatcacctagccacactgatccctattttgatatgccttcaatgcccggctttatatgtttttcaaagttacataataagagaaagaaaaaagtacactaatggaacatatttctgctttgttttcaaaatgtgttctcttcaagttactggtccaaagtaccttgtttggtaggaacccagacaattctctaatgttgctcttacctctccagaatatatttttctaatgtgagtttcttttcttttgcagcatatttgtgtcattagtgaaactgggaaagccaaatacaaagcctaagatgagagttcaagttgaatctgcaaacacgaggagtcccattcatgttcccagtaaaattaccaagcattctagttctgcagccatctgcttagtacagctttttgcatgaaccttctaagaattttatggttttttatttttagaaatgtcagttgctgcattcttaaactttttatttgcactatgagccttcgatagattgtcgcttaccttgtgagcaaagtaaatctcttaaattaccactattaaaaatgctgagattgtatctgtaagaaacatttaaagaggagatatattagttttttaattggtattttaatttttatatattcgggaaagaaagctgtgattgaatattgttaattataccaccatgtgtttagaacagtgtgaagcactcagtttcatgtcagtaaccatctaagaagttttgctcagtggaataaacatatgttttatcagtgttgcctatattttcccacttggattctttcagtgagtcattgtcaacagttcctttaaatgcaaataaattctaaaaattta

SEQ ID NO: 98 - DNA, 300 bp, Cricetulus sp.
  source 1..300: mol_type = genomic DNA
gacccatttgcattctgaactgggaaatacagagtcatgaaggagactcagaccaaagtgaccacagcctacgttgctggtttaccctcaaacagagacagcgaaggaaggatctcaggtgtcctctccactaagctctgtggggcagcaggtgcagggaaaaaaaattatatatatataccctcaacaagcaagtacaacatgtctccacccgaatccacctgaaggctttgaataatagacaggggagtccactgcctagtcctggtctcaggcctggaagtgtttcccaatcttttc

SEQ ID NO: 99 - DNA, 1254 bp, Cricetulus sp.
  source 1..1254: mol_type = genomic DNA
tcggaagcagtttgcatttgtggtactttgagtgttttcctagaaaaatgaaaaagcaggtgaagtgtgtgagcttggcgtgcgttccaataggccagcagtagaacttgcgtgggcatttaaattgctaaatgtgttacttttaaggagaaatcttaagtttgtttcttgttgacaatctaaggtcttagtagcctctttcctaagccccagtaggtttctcttttaccaacccaataacgaaaattgctaacgaagtatttttgctttggaattataggacaatagtggacctggaggagcacacttgagttctggtggcagggattattttgagactaccctagtttagtggttttgtttttagcataactattactaattcagtaaacttagtatgctctcagtaccactttaaggtagatttgccctatatcttaaaacgtttttttgcttttcctaggatcaaaggtgtgaacgtaccgtacccctcgtggaagagactaaaagataaaattgtatttacaggtaagtctgcaccttaaaaagcctgtcctggttttactggacaactactaactttttaccatggagtaatttattttggaataaaatgttgaatcaacactggctaaacctacagaacttggcctggtatgcctttagttccagcatttgggaggccaaggcatgcagatctctttgatatcgaggtcagcttcacctgcagatctagccagagctatagagaaatcctgtctggaaaataatctagaaaactaaaaacaaacttcccccaaatgaaacccttacacagtggatttgatgacaaagcttaaataatatcgttggcatggtgcctaaaagcacttactacttacttttttattcatgccttagtaaaataggggagagatgattgtgggggcagtgttggaactctgggcctgattgctaccctgggagaggttatattagttggtgaattaatgttacctaggcagaggctagctcttaatttgtggttggaatgtagtaccagtgactttttcttttatttttatttttttttgaaaatttgttttatgagcataagttgtgcccatttgtaagagtacttggtgtgtggaggcgagggcgtgggtcccctagaactgacattacaggcaattgtgtatctaccatgttggtgttgggacatgaacccaggtcctcttatagggccacaagtactcttaactgttgagtcatgtc

(19) United States
(12) Patent Application Publication
(10) Pub. No.: US 2024/0141385 A1
(43) Pub. Date: May 2, 2024

(54) TUNABLE TRANSPOSON SYSTEMS

(71) Applicant: Aridis Pharmaceuticals, Inc., Los Gatos, CA (US)

(72) Inventors: Kathy Ngo, San Jose, CA (US); Jennifer Woo, La Mesa, CA (US); Binh Pham, Mountain View, CA (US); Vu Truong-Le, Campbell (CA)

(21) Appl. No.: 17/811,414

(22) Filed: Oct. 27, 2022

(51) Int. Cl. C12N 15/90; C07K 16/00; C12N 9/22; C12N 15/11; C12N 15/86

(52) U.S. Cl. CPC C12N 15/907; C07K 16/00; C12N 9/22; C12N 15/11; C12N 15/111; C12N 15/86; C07K 2317/14; C12N 2310/20; C12N 2750/14143; C12N 2800/30; C12N 2800/80

(57) ABSTRACT

This invention provides methods and systems for enhancement of protein production from mammalian cell lines in a drug inducible manner. The methods described herein can be used to generate a protein production cell line wherein the gene coding the protein product of interest is inserted into specific safe harbor loci (SHL) within the cell's genome and the gene copy number is induced to amplify by the use of an antibiotic inducer. The method enables for the conditional activation of the drug inducible transposase. The drug inducible gene amplification method described herein effectively functions as a molecular dial: combining drug-inducible homologous recombination and conditional gene activation to fine-tune gene amplification in mammalian systems.

### FIELD OF THE INVENTION

The inventions are in the field of biological expression systems. Aspects include systems for controllable incorporation of exogenous genes into expression host cells by transposons. The systems include features working in combination to control transposon-mediated gene insertion locations, control gene copy number, and provide genetic stability to the cells. The systems and associated methods allow functions to be switched on or off with fast acting molecular signals.

### BACKGROUND OF THE INVENTION

Protein-based products have emerged as important biopharmaceuticals that treat human diseases. These drugs are predominantly engineered to be produced by mammalian cell lines since these cells often produce high quantities of therapeutic proteins with appropriate critical quality attributes (CQAs) that impact potency and immunogenicity. High volumetric productivity, product titer and stability are important criteria to obtain efficient production of protein therapeutics. These features may be brought about by a combination of changes that collectively make the host system a high protein producer. These attributes include high translation efficiency, gene copy number, secretory capacity, growth capacity, duration of viability at maximum cell density, and post-translational modifications. To date, improvements in protein production have been achieved by media and bioprocess optimization such as feeding strategies and process parameter controls that are both time consuming and need to be empirically defined. Genome engineering tools to maximize productivity by gene amplification are also being applied (Fischer, 2015). The prior arts used to amplify the gene of interest lack precise control of the number of gene copies that is amplified, lack control of location of gene insertions of the amplified gene, and lack predictable genetic and cell line stability. Four key desirable attributes are covered by the invention which include, e.g., control of copy number of the gene of interest, control of the insertion site into the host cell genome, targeted placement of the insertion site into the host cell genome, and genetic stability of the inserted gene for consistent productivity.

Genetic stability of the inserted gene is a major concern in the field. Traditionally, in order to generate CHO cell lines with high levels of productivity of the protein of interest, gene copy amplification of the inserted gene is performed by use of a cytotoxic drug-induced gene amplification method involving the amplification and selection for high copy number of the dihydrofolate reductase (DHFR) gene under the selection pressure of increasing concentrations of methotrexate (MTX). As MTX is an inhibitor of DHFR, only those cells with high copy number of DHFR survive. This DHFR/MTX method has been used extensively in the manufacturing of antibodies. The glutamine synthetase (GS)/methionine sulfoxamine (MSX) system is similar to the DHFR/MTX system. The GS enzyme catalyzes the production of glutamine from glutamate and ammonia. Methionine sulfoxamine binds to the GS enzyme and prevents the production of glutamine. Gene amplification occurs when cells are subjected to increasing concentrations of MSX. One drawback of the DHFR/MTX and GS/MSX method is that it is labor-intensive and obtaining high-producer clones requires repeated cycles of selection and cloning that can take 3 to 4 months for each successive cycle. Furthermore, high-producer clones obtained by these methods are frequently unstable and show a rapid decrease in protein synthesis as cell culture time progresses. Methods to address genetic instability include the use of the mammalian replication initiation region (IR) and a matrix attachment region. This has been shown to generate stable clones in contrast to DHFR/MTX or GS/MSX system. The IR/MAR system is a gene amplification method based on using a plasmid bearing a mammalian replication initiation region (IR) and a matrix attachment region (MAR), which results in the spontaneous initiation of gene amplification in transfected cells and has been used in combination with DHFR/MTX to generate stable productive cell lines (Noguchi, 2012). Both IR/MAR and GS/MSX systems offer improvement on time to generating genetically stable clonal cell line selection and generation over DHFR/MTX but neither system has control over integration site or gene copy number. However, all of the three described selection methods are laborious and time consuming with lack of control over both integration site number and gene copy number. In the above-mentioned systems, gene copy and integration site number are a pure stochastic process.

In an effort to improve on these methods, an alternative form of gene amplification is the use of transposable elements (TE). TEs have shown great promise in recent years for multi-site integration of transgenes and their use is enhanced by the availability of molecular genetic tools. Two classes of TEs have been identified according to their mechanism of transposition, which can be described as either copy and paste (Class I TEs) or cut and paste (Class II TEs). Class I TEs are copied in two stages: first, they are transcribed from DNA to RNA, and the RNA produced is then reverse transcribed to DNA. This copied DNA is then inserted randomly back into the genome at a new position. The reverse transcription step is catalyzed by a reverse transcriptase, which is often encoded by the TE itself. These class I TEs are also called retrotransposons. In contrast, the ‘cut-and-paste’ transposition mechanism of class II TEs does not involve an RNA intermediate but requires transposase enzyme and inverted terminal repeats (ITRs). The transpositions are catalyzed by several transposase enzymes. Some transposases non-specifically bind to any target site in DNA, whereas others bind to specific target sequences. The transposase makes a staggered cut at the target site producing sticky ends, cuts out the DNA transposon and ligates it into the target site. A DNA polymerase fills in the resulting gaps from the sticky ends and DNA ligase closes the sugar-phosphate backbone. This results in target site duplication so that the insertion sites of DNA transposons may be identified by short direct repeats (a staggered cut in the target DNA filled by DNA polymerase) followed by inverted terminal repeats (ITRs) which are important for the TE excision by the transposase. The advantages of transposon methods and differences from other gene amplification methods is summarized in a comparison of gene amplification methods currently in use in Table 1.

TABLE 1
Gene Amplification Technology Comparison

| | DHFR/MTX | GS/MSX | IR/MAR | Transposon |
|---|---|---|---|---|
| Maximum copy | 10 | 10 | 10 | 50 |
| number | | | | |
| Time to | >6-9 | 1-2 | 1-2 | 1-2 |
| amplification | months | months | months | months |
| Control of copy | No | No | No | No |
| number | | | | |
| Control of | No | No | No | No |
| integration | | | | |
| Preferential | No | No | No | Yes |
| targeting to safe | | | | |
| harbor locus | | | | |
| (SHL) | | | | |
| Stability | Few stable | Few stable | Few stable | Few stable |
| | clones | clones | clones | clones |
| Drug | No | No | No | Yes |
| inducibility | | | | |

DHFR/MTX dihydrofolate/methotrexate; GS/MSX glutamine synthetase/methionine sulfoxamine; IR/MAR: initiation region/matrix attachment region.

Transposons allow for packaging and stable integration of large transgenes. However, limitations with this current technology exist. The transposition process occurs in a stochastic fashion, resulting in transgenes integrating randomly. While there are hotspot integration sites, transposons may also integrate in developmentally relevant genes, transcriptional regulatory genes, super-enhancers, and oncogenes or those involved in cellular metabolic pathways thus posing a safety concern for bio-manufacturing (Balasubramanian, 2016). Furthermore, the current transposon technologies have limited control over gene copy number and integration site number. Table 2 summarizes the features of the current transposon technologies that include PiggyBac (EP2401376), Leap-In Transposase (U.S. Pat. No. 9,418,767), and Sleeping Beauty Transposase (U.S. Pat. No. 7,160,682) and their limitations. It is also noted that genetic stability is not controlled. Not captured in this table is the low efficiency of transposase delivery by protein or mRNA resulting in re-excision events and re-integration events which can also occur randomly and likely contribute to the inconsistent genetic stability observed.

TABLE 2
Transposon Technology Comparison

| | PiggyBac | Leap-In | Sleeping Beauty |
|---|---|---|---|
| Efficiency and Titer | 4-11-fold | Up to 3-fold | Poor efficiency; |
| | increase | increase | up to |
| | | | 10-fold |
| | | | increase |
| Control of copy | Yes | No | Yes |
| number | | | |
| Preferential targeting | Hotspots | Not | Integration |
| to safe harbor locus | occurs in | demonstrated | hotspots |
| (SHL) | oncogenes | | preferential |
| | | | to SHL |
| Probability of Clonal | Low | Low | Low |
| Stability | | | |
| Drug inducibility | No | No | No |
| Footprint free editing | Yes | Yes | Yes |
| Cargo capacity | 20 kb | Not | 12 kb |
| | | demonstrated | |
| Transposase Delivery | mRNA/ | mRNA/ | mRNA/ |
| | Protein | Protein | Protein |

While the current transposon technology shows improved genetic stability over the existing DHFR/MTX, GS/MSX, and IR/MAR gene amplification technologies, there is still a need for technology with better control and predictability in gene insertion. Lack of predictability is likely associated with genomic chromatin states and chromatin structural proteins that may be poorly characterized but are associated with and may influence transposon insertion preference (Yoshida, 2017).

The disadvantages in the current state of art transposon technology can be summarized as follows
Currently, delivery of transposase in the form of plasmid, mRNA, and protein are performed by electroporation which is inefficient and lacks targeted placement to specific areas within the genome of the target host.
The number of integration sites is random and therefore does not avoid targeting oncogenes or developmentally relevant genes in the genome.
No control in integration site number or gene copy number during the amplification process is available
In vitro delivery of protein, mRNA or plasmid form of transposase can result in long-term activation leading to poorly controlled re-integration or re-excision events.
Accordingly, the technology requires the use of excision-deficient mutant of transposase to prevent re-excision and re-integration.
The number of gene copies that are amplified by said transposase can only be determined at the single cell level and performed during the clonal selection process.
Inducible gene expression systems have been used to control the amount of gene product that is transcribed. However; there is no existing technology that can precisely control gene copy number or integration site number in a dosage dependent manner.

As summarized in Tables 1 and 2, there is a need for a gene amplification method that provides fine-tuned control of integration site number and gene copy number, and precise targeting to genomic loci that does not alter the physiological state of the cell while delivering a high titer protein production cell line suitable for biomanufacturing processes. The ability to control for inserted gene copy number, targeted insertion sites, targeted placement within the host cell genome, and genetic stability of the gene of interest are key areas enabled by this invention.

In view of the above, we believe a need exists for a transposon expression system that eliminates unpredictable changes to the cell lines associated with random integration. It would be desirable to obviate the need for delivery of transposase enzyme into the cell. Benefits could also be realized from an ability to control the timing and amount of transposase that is expressed or delivered into an expression host cell line. A transposon based expression system that eliminates re-excision and reintegration events would reduce unpredictable changes. The present invention provides these and other features that will be apparent upon review of the following.

### SUMMARY OF THE INVENTION

The systems and methods described herein provide gene amplification utilizing, e.g., a small molecule inducer turning on rapid gene amplification that can take less than a day. Unlike the DHFR/MTX approach, this method does not require constant exposure of MTX which can take up to 4 to 6 months for stable clonal section (see Table 1). Our systems and methods eliminate the stochastic nature of current gene amplification systems through targeted gene amplification to safe harbor loci (SHL), and provide precise control of gene amplification and integration site number by means of regulating the activity of an inducible transposase that only rearranges to the correct orientation after exposure to an antibiotic. For example, using a fast-response antibiotic such as Doxycycline (Dox), a site-directed knock-in can provide rapid gene amplification, e.g., combined with efficient clonal selection. Such procedures can complete identification of lead clones, e.g., in as few as 3 to 5 days.

One embodiment of the invention provides a modified Sleeping Beauty transposase engineered in a tripartite system that combines a drug-inducible system, recombinase, and two sets of heterotypic sites arranged in a unique orientation to allow for calibrated control of gene amplification.

Furthermore, by placing the rearrangement of the transposase under the control of a synthetic promoter, activation of a downstream gene can be made sensitive to antibiotic exposure. By combining all elements this invention is essentially a tunable molecular machine that allows for fine-tuned control of gene copy number, control of integration site number, site-specific gene integration to safe harbor locus, and genomic stability.

Our described technology improves on the current transposon technology allowing for preferential targeting of transposons to safe harbor loci (SHLs) of choice. This is done by conditionally predisposing a transposon to target a chosen SHL sequence. Alteration of the epigenetic memory of cells can allow genetic reprogramming of a cell, e.g., resulting in pluripotency of differentiated cells. One embodiment of the invention provides for reprogramming of the transposon machinery to integrate specifically to SHL.

The systems and methods of the invention employ a master regulatory construct and an expression construct, e.g., configured to enable precise control of an expression transposon insertion locations and copy number. The invention makes novel use of inducing agent controlled Cre/Lox inversion and excision activities to turn on, turn off, and/or modulate a specific functionally interacting expression transposon in a host cell. The systems and methods can provide error free insertion of the expression transposon at safe harbor loci in controlled numbers, while maintaining an ability to activate or deactivate expression of a protein of interest.

The systems for conditional control of transposon copy number in an expression host cell typically comprise a master regulatory construct and an expression construct. The master regulatory construct and expression transposon construct can be on the same nucleic acid in the host cell, or on different nucleic acid strands.

The master regulatory nucleic acid construct can include a) a transposase sequence in reverse orientation downstream from a promoter sequence, which promoter sequence is controlled by an inducing agent; and b) a Cre recombinase sequence with expression fine-tune control of inducing agent. Meanwhile, the transposase sequence is upstream from a reverse oriented heterotypic first Lox sequence and downstream from a second Lox sequence forward oriented between the promoter sequence and the transposase sequence, this configuration allowing inversion of the transposase sequence for functional expression. The master regulatory constructs in themselves can be considered novel tools, useful and functional in tunable control of expression transposon constructs generally.

The expression transposon nucleic acid construct can include a) a transposon sequence encoding a protein sequence of interest flanked by inverted terminal repeats (ITRs) that act as binding targets of the transposase, and b) a binding site (BS) for the first transcriptional activator upstream from the ITRs.

In the presence of an inducing agent, such as doxycycline, Cre expression is induced, resulting in CRE/Lox inversion of the transposase to forward orientation allowing functional expression of the transposase. When combined with Cas9 and site-specific gRNA, it enables specific insertion of the transposon at particular safe harbor loci (SHL) of interest for expression of the protein of interest.

In particular embodiments, the transposase can be a Sleeping Beauty transposase. The promoter sequence can be a TRE3G promoter and the inducing agent is Dox/rtTA. A protein of interest can be any polypeptide, such as antibody heavy chain and/or an antibody light chain. A preferred host cell is Chinese hamster ovary (CHO), with acceptable alternate cell lines including, e.g., SP2/0, NS0, HEK, and 293T.

In many embodiments, insertion of the transposon is enabled by knock-in into the SHL by AAV and CRISPR enzymes, e.g., Cas9, Cas12a (Cpf1), Cas12b, CasX, or CasY.

The master regulatory construct can include a ubiquitous promoter controlling expression of the rtTA (reverse tetracycline-controlled transactivator protein) and a selective pressure resistance factor, such as Blasticidin (Bsd) resistance. Additional ubiquitous promoters can include EF1a, CAG, Cbh, SV40, UBC, CMV, EFS, CMV, and/or the like. In a preferred embodiment, the ubiquitous promoters are CMV and EF1a promoter elements.

The inducing agents of the constructs can be a transcription activator (TA), such as GAL. Alternately, the inducing agents can comprise a ligand/receptor inducible system, such as, e.g., a combination of doxycycline (Dox) and reversible tetracycline transactivator (rtTA). In preferred embodiments, master regulatory constructs are controlled by ligand/receptor inducing agents, e.g., wherein the ligand is a small molecule or drug, facilitating control by external introduction of the ligand to the host cell.

The methods for conditional control of transposon copy number in an expression host cell typically comprise provision of a master regulatory construct, an expression construct, and steps controlling interactions of the constructs' features.

A method for conditional control of transposon copy number in an expression host cell can include, e.g., a) providing a nucleic acid master regulatory construct, as described herein; b) providing an expression transposon nucleic acid construct, as described herein; c) applying the inducing agent to the cell, thereby inducing expression of Cre; d) inverting the transposase to forward orientation by a CRE/Lox inversion, thus allowing expression of the transposase; e) inserting the transposon at one or more safe harbor loci (SHL) via the use of Cas9 and site-specific gRNA; and, f) expressing the protein of interest in the host cell from the one or more SHL sites. In certain embodiments, controlling the copy number of inserted transposons is accomplished by adjusting the concentration of the inducing agent or receptor ligand.

In other aspects, the promoter sequence is a TRE3G promoter and the inducing agent is Dox/rtTA; the host cells are stabilized in culture with selective pressure, e.g., based on blasticidin (Bsd) and/or II puromycin (Puro) resistance; and transposon insertion into the host cell genome SHLs is by knock-in using AAV and by CRISPR/Cas9, Cas12a (Cpf1), Cas12b, Cas13, Cas14, CasX, or CasY. In one useful embodiment, the protein of interest encoded by the transposon is an antibody protein.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification, the singular forms “a”, “an” and “the” can include plural references unless the content clearly dictates otherwise. Thus, for example, reference to “an activator” includes a combination of two or more activators; reference to “bacteria” can include mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

As used herein, the term “transposon” refers to mobile segments of DNA that can move from one locus to another, as understood in the art.

The term “endogenous”, as used herein, refers to those moieties native to a cell, as compared to exogenous moieties, as understood in the art. For example, an endogenous gene is a gene originally in a host cell before it is modified by receipt of extraneous nucleic acids, e.g., by electroporation, genetic engineering, transfection and/or the like.

The term “inducing agent” as used herein, refers to chemical ligands, small molecules, antibiotics, protein, fusion protein or conditional triggers such as light and temperature, that when added to a biological system, results in activation or repression of target genes. Inducing agents can include ligand/receptor combinations, e.g., a drug or small molecule in combination with transcriptional activators (e.g., Dox/rtTa). Inducing agents can include but are not limited to tamoxifen, cumate, quinic acid, doxycycline/rtTA, tetracycline, 4-hydroxytamoxifen (4HT), photoactivatable molecules (e.g. Cry2/CIB1), rapamycin and its analog, AP21967, and/or the like.

Transcriptional activators, as used herein, are polypeptides that bind to regions of DNA in the promoter region and recruit RNA polymerase, e.g., at a location upstream from the 5′ end of the transcription start site and activate transcription of downstream target genes. Such transcriptional activators can require binding of a small molecule or drug ligand in order to take on the transcription activation function.

The term “ubiquitous promoter”, as used herein are as commonly known in the art. Ubiquitous promoter sequences are DNA sequences (typically 100 to 1000 base pairs) that promote binding of RNA polymerase, e.g., at a location upstream from the 5′ end of the transcription start site, resulting in high level of gene expression in many cell types.

The term “heterotypic recombination sites”, as used herein refers to recombination sites that are not identical and when placed in the same or opposite orientation do not result in homologous recombination. Recombination sites used herein are DNA sequences consisting of palindromic recognition regions and a spacer region.

The term “forward orientated” as used herein refers to, e.g., DNA sequences placed in 5′ to 3′ direction, in the same direction as its upstream promoter sequences.

The term “reverse orientated” as used herein refers to, e.g., DNA sequences placed in 3′ to 5′ direction, in the opposite direction as its upstream promoter sequences.

The term safe harbor locus (SHL) used herein is commonly known in the art. SHL are genomic loci where genes or other genetic elements can be safely inserted and expressed without altering the cell physiological state. SHL are further described as genomic locations where new genes or genetic elements can be introduced without disrupting the expression or regulation of adjacent genes.

As used herein, the term “master transcription regulatory elements (MTRE) activation” refers to, e.g., methods of transcriptional activation of Prdm1, Irf4, and Xbp1 using nuclease-dead CRISPR enzymes, Cas9, Cas12a (Cpf1), Cas12b, Cas13, Cas14, CasX or CasY fused to transactivation domain of VP64 (dCas9-VP64), accessory fusion protein: MS2-p65-HSF1, and nine sgRNAs that target regions of DNA flanking the transcriptional start site and downstream promoter elements of the above mentioned genes.

As used herein, the term inversion refers to recombination events in which two identical recombination sites are in the opposite (or “reverse”) orientation flanking a sequence, resulting in the inversion of the DNA sequence.

As used herein, the term excision refers to recombination events in which two identical recombination sites are in the same orientation flanking a sequence, resulting in the excision of the sequence, deleting it from its original locus.

As used herein, the term CRISPR refers to Clustered Regularly Interspaced Short Palindromic Repeats. CRISPR is a notoriously well-known complex of any of the following Cas enzymes: Cas9, Cas12a (Cpf1), Cas12b, Cas13, Cas14, CasX, or CasY (having a nuclease activity) and a guide CRISPR RNA (gRNA or crRNA). The combination can target the nuclease activity to a precise location on a DNA strand. dCas9 (dead or disabled Cas9) is lacking the nuclease activity but retains the specific targeting ability in combination with a gRNA.

gRNA (guide RNA), as used herein, is as commonly known in the art. The gRNA is a short RNA composed of a scaffold sequence necessary for Cas protein binding interaction and a spacer sequence (18-20 nucleotides) that defines a DNA target to be modified.

The term targeted insertion or knock-in used herein refers to, e.g., the combined use of CRISPR Cas enzymes (e.g., Cas9, Cas12a (Cpf1), Cas12b, Cas13, Cas14 CasX, or CasY), gRNA/crRNA, and donor cassette to stably introduce a foreign DNA sequence at a defined genomic location in a cell.

### BRIEF DESCRIPTION OF THE DRAWINGS

FIGS. 1A to 1C are schematic diagrams describing a stable cell line generation process for antibody gene amplification using iSB technology. In FIGS. 1B and 1C, master regulatory construct (FIG. 1B) and transposon expression constructs (FIG. 1C) are shown using an inducible Sleeping Beauty gene anplification system for mAb production enhancement.

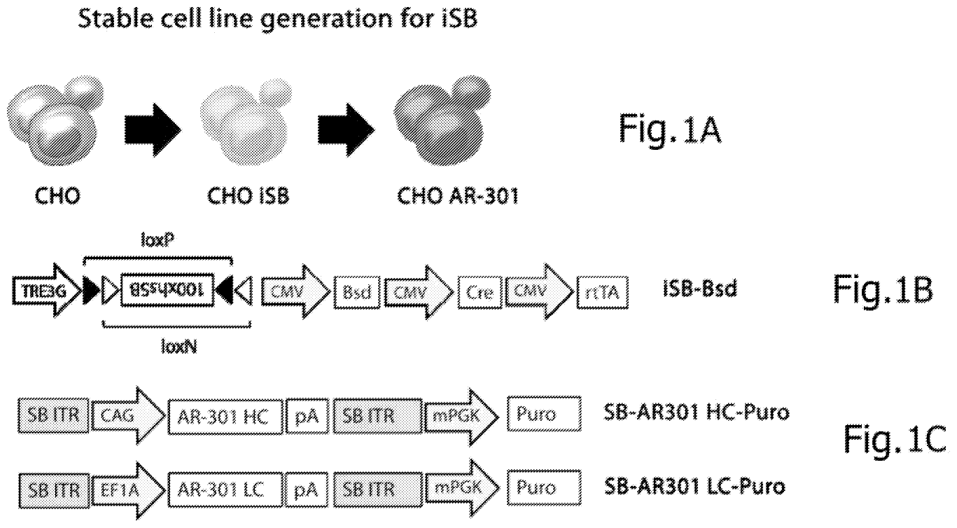

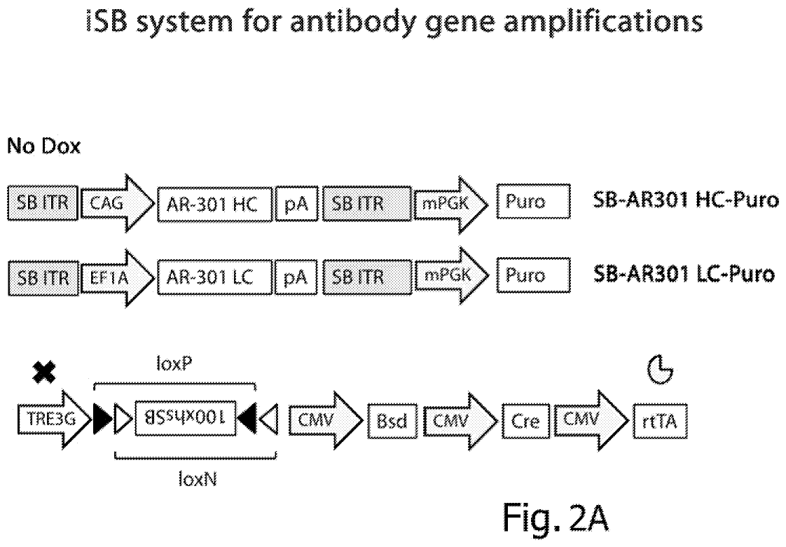

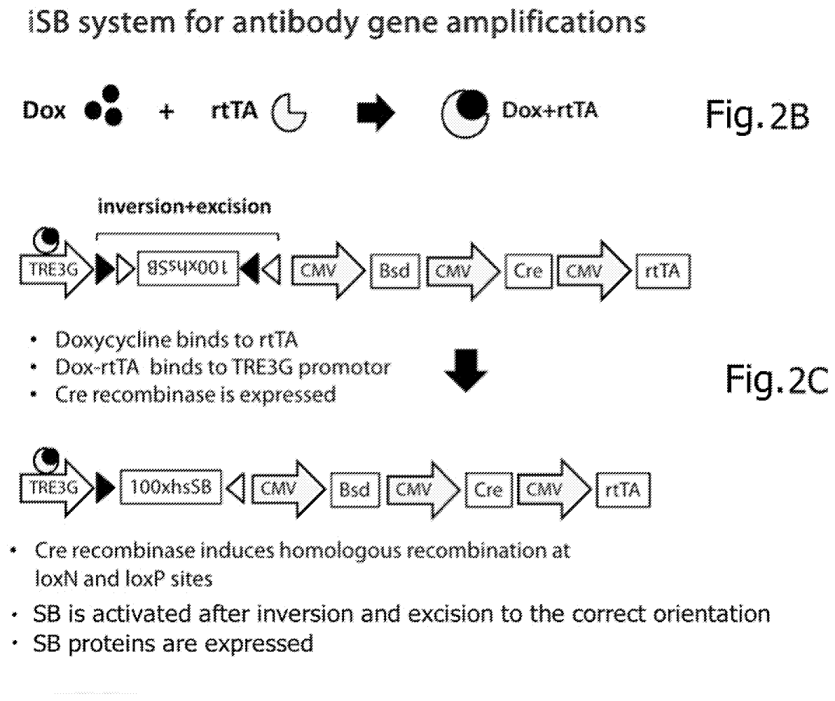

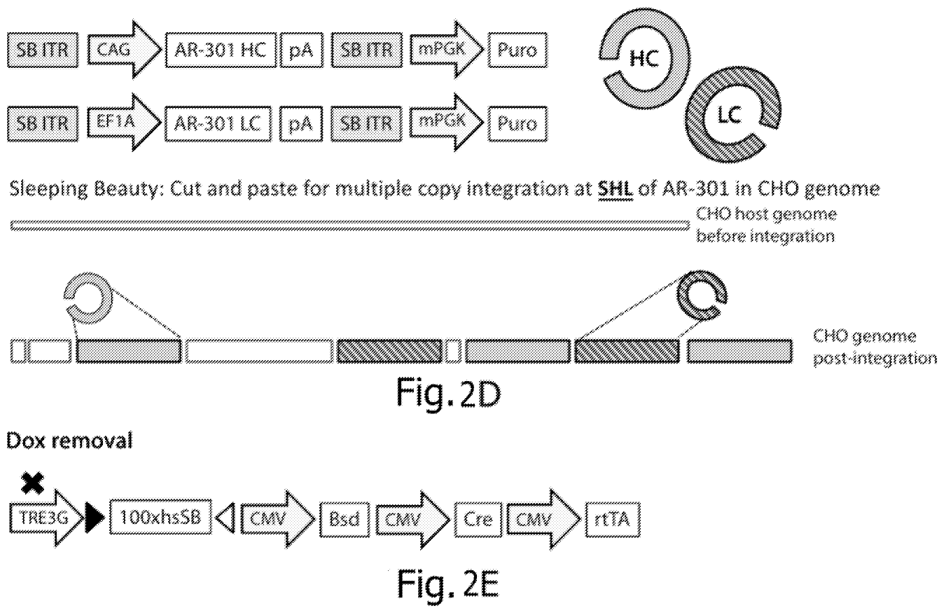

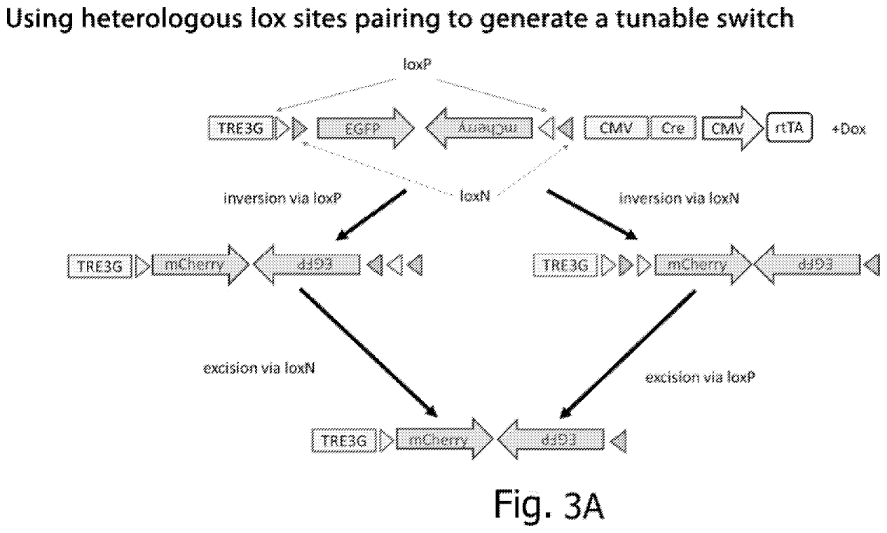

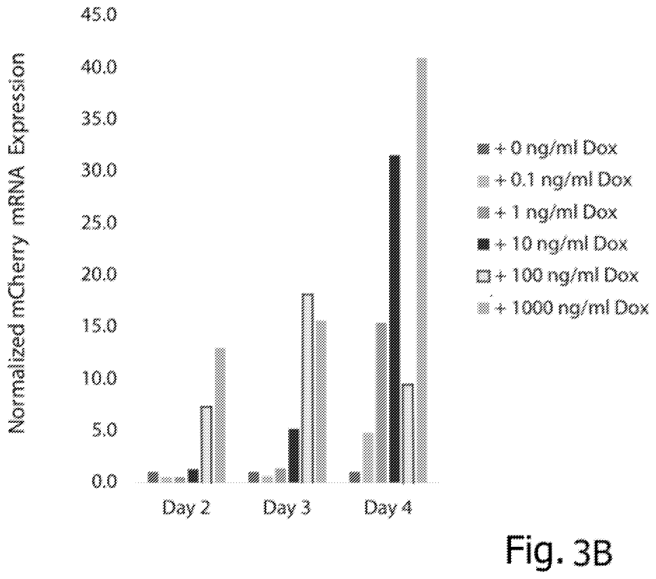

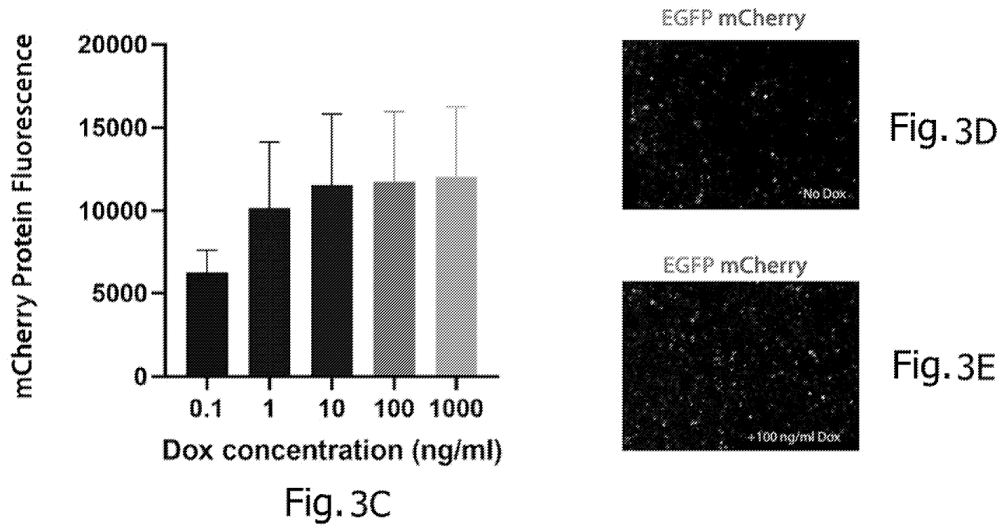

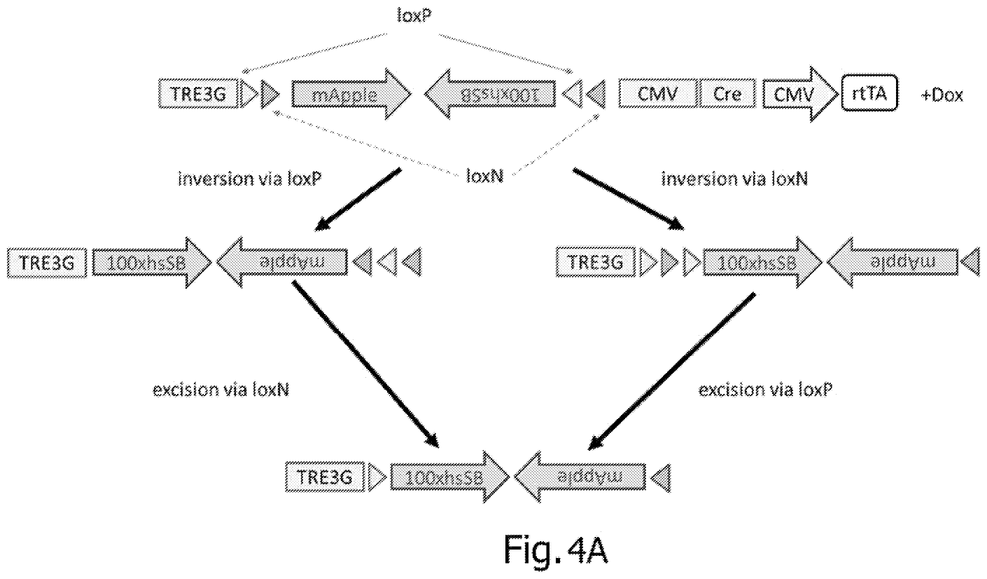

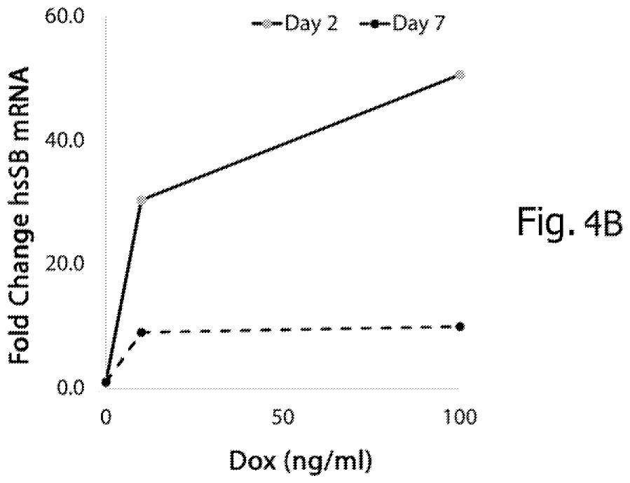

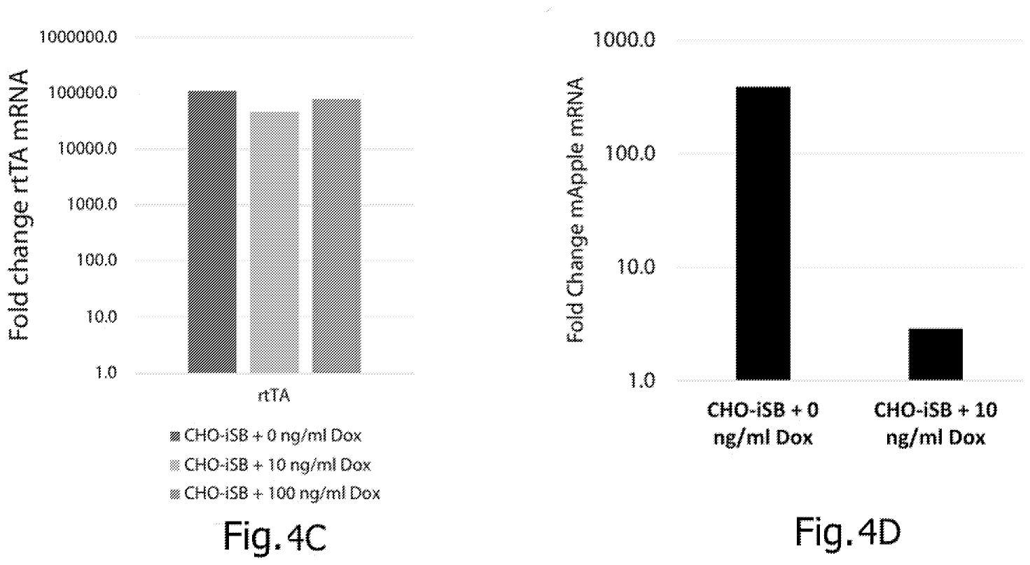

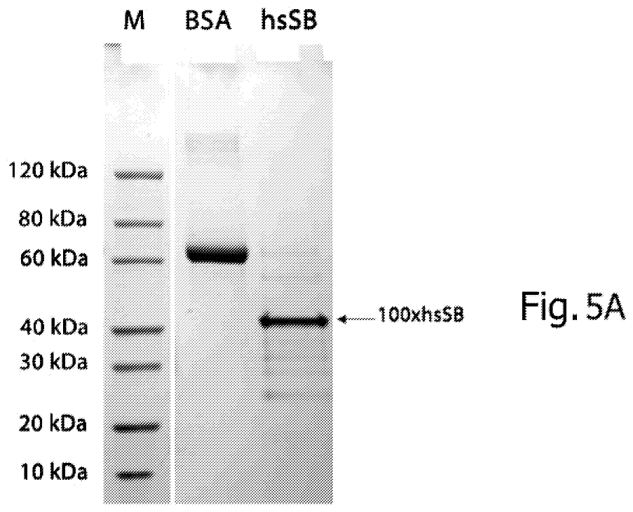

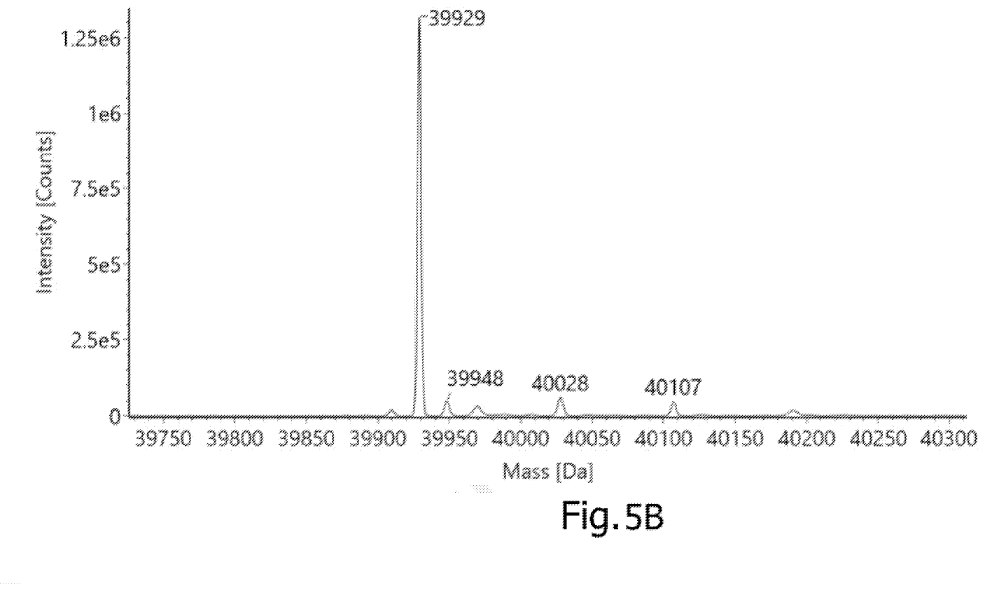

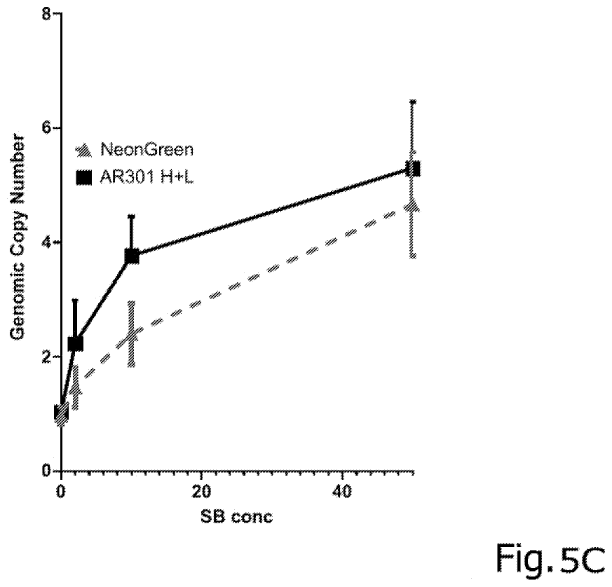

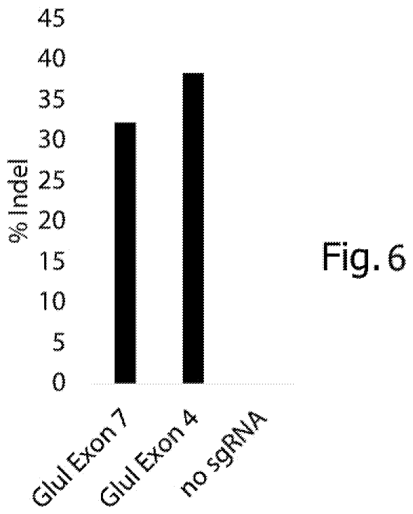

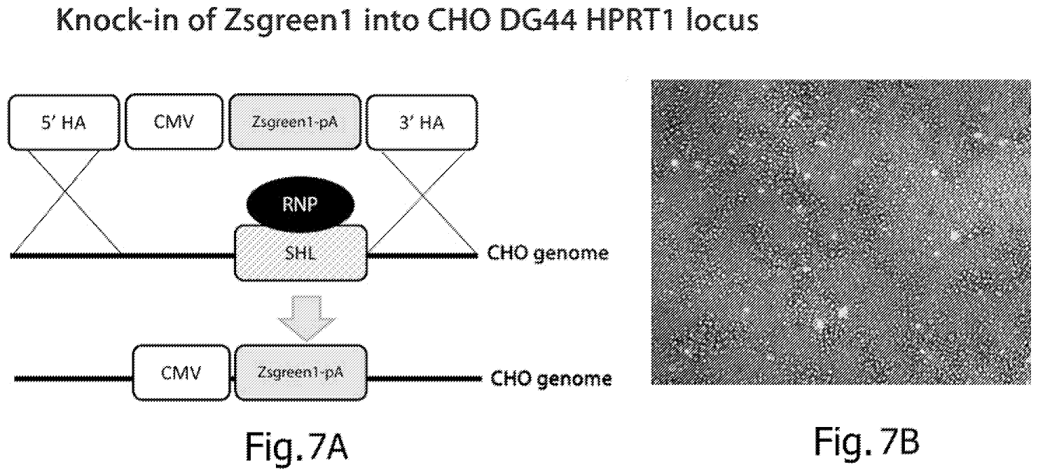

In FIG. 1A, a parental CHO cell line (labelled CHO) was modified by insertion of a master regulatory construct such that the cell line CHO-iSB was generated (labelled CHO-iSB). Following stable cell line generation of CHO iSB, the final cell line CHO AR-301, was generated by insertion of an expression construct. In this example, CHO-AR-301 contains both the master regulatory construct and the expression construct. The expression construct consists of a heavy chain gene (AR-301 HC) and a light chain gene (AR-301 LC) that in combination encode for a monoclonal antibody (mAb) against S. aureus HLA (AR-301).

In FIG. 1B, the master regulatory construct, iSB-Bsd, contains a tetracycline response element and a third generation (TRE3G) promoter with two pairs of heterotypic lox sites in cis: loxP (dark triangles) and loxN (empty triangles). The loxP and loxN are placed in opposite directions to activate highly soluble Sleeping Beauty (100×hsSB) transposase upon addition of Doxycycline (Dox). The 100×hsSB (boxed) is placed in reverse orientation in-frame to the TRE3G promoter. A sequence encoding a Cre recombinase, reversible tetracycline transactivator (rtTA), and Blasticidin (Bsd) resistant genes are driven by CMV promoter (arrow).

FIG. 1C shows a transposon expression constructs for amplification of heavy and light chain genes of mAb against S. aureus HLA (AR-301). Expression of the antibody heavy chain (HC) gene is driven by a CAG fusion promoter (arrow) and the light chain (LC) gene is driven by a EF1A promoter (arrow), in cooperation with SV40 poly A tail (pA). The puromycin (Puro) resistant gene (boxed) is driven by mouse phosphoglycerate kinase 1 (mPGK) promoter (arrow); SB ITR: Sleeping Beauty Inverted Terminal Repeats

FIGS. 2A to 2E provides step-wise schematic diagrams of an inducible sleeping beauty (iSB) system utilized for antibody gene amplification and expression. The figures present a flow diagram for a system enabling “OFF” and “ON” status of integrated constructs. The system consists of the master regulatory construct, iSB-Bsd and the transposon expression constructs: SB-AR301 HC-Puro and SB-AR301 LC-Puro as outlined in general above for FIGS. 1B and 1C.

FIG. 2A shows schematic vector constructs for activation of the Sleeping Beauty System. The top schematic is the transposon expression construct of the heavy and light chain genes of AR-301 mAb, containing TA-rich Sleeping Beauty transposon inverted terminal repeats (SB ITR) at the 5′ and 3′ end; while the bottom schematic is the master regulatory construct (iSB-Bsd) without the addition of the inducing agent, Doxycycline. The loxP and loxN are placed in opposite directions to activate highly soluble Sleeping Beauty (100×hsSB) transposase upon addition of Doxycycline (Dox). The 100×hsSB (box) is placed in reverse orientation in-frame to the TRE3G promoter. A sequence encoding a Cre recombinase (Cre), reversible tetracycline transactivator (rtTA), and Blasticidin (Bsd) resistant genes are driven by the CMV promoter (arrow). Without the antibiotic ligand doxycycline (Dox), TRE3G is off (black X) and only rtTA (receptor—partial gray circles) and Bsd are expressed. The rtTA by itself cannot bind to the TRE3G promoter to activate downstream target genes. Additional abbreviations: pA, polyadenylation signals from simian virus 40 (SV40); Puro, gene encoding for Puromycin resistance.

As shown in schematic in FIG. 2B, when Doxycycline ligand (black circle) and reversible tetracycline transactivator (rtTA, gray partial circle) is expressed in the same cell, Dox binds to rtTA. When the Dox-rtTA complex is recruited to the TRE3G promoter, it activates downstream target genes in a dosage-dependent manner.

FIG. 2C demonstrates the induction events following Dox addition (black circles). With the addition of Dox, rtTA will bind to Dox and form a Dox-rtTA complex that is recruited to the TRE3G promoter. The TRE3G promotor combined with Cre expression will induce homologous recombination at compatible lox sites (loxP and loxN) resulting in activation of the SB by inversion and excision to the original in frame orientation. This sequence of induction events following Dox addition results in expression of hsSB proteins.

In FIG. 2D expression of hsSB proteins from FIG. 2C plus the transposon expression construct containing heavy and light chain genes of AR-301 allows for type 2 “cut and paste” amplification of AR-301 HC (solid gray box) and AR-301 LC (gray box with dashed lines) genes and integration into a safe harbor locus (SHL) of the host genome (white). This results in multiple copies of the HC and LC genes in the genome.

In FIG. 2E, removal of Dox turns off expression of SB and high-titer stable cells containing amplified heavy and light chain genes of AR-301 are selected for Bsd and Puro resistance. No re-integration events will occur as transposase is no longer active.

FIGS. 3A to 3C demonstrate the tunable switch of proteins in the master regulatory construct and confirmed using fluorescent protein reporters (mCherry and eGFP).

FIG. 3A shows schematic diagrams applied in sequence to create a tunable switch of proteins. mCherry fluorescent protein and EGFP fluorescent protein were chosen to visualize the tunable switch. The gene encoding for mCherry fluorescent protein is placed in reverse orientation in-frame to the TRE3G promoter while EGFP fluorescent protein is placed in the forward orientation in-frame to the TRE3G promoter. A sequence encoding a Cre recombinase, reversible tetracycline transactivator (rtTA), and Blasticidin (Bsd) resistant genes are driven by CMV promoter (arrow). Addition of Dox results in inversion and excision event, resulting in loss of EGFP and expression of mCherry protein.

FIG. 3B shows mCherry mRNA induction and mRNA expression occurs in a dosage dependent manner by Dox. The mCherry mRNA was measured by RT-qPCR in stable cell line at Day 2, Day 3, and Day 4 expressing a tunable switch as described in FIG. 3A. Tunable induction is demonstrated by use of a titration of varying levels of Dox used for induction. Levels of Dox tested ranged from 0 ng/ml, 0.1 ng/ml, 1 ng/ml, 100 ng/ml, and 1000 ng/ml. Dox exposure was maintained for 8 hrs at Day 0. The mCherry expression data is normalized to a housekeeping gene, EIF3I and the Day 0 timepoint.

FIG. 3C shows an increase of mCherry protein expression occurs with increasing Dox concentration used as measured by mean fluorescence intensity of mCherry. The mCherry protein fluorescence was measured using 10,000 cells per condition at Day 4 following the tunable switch as described in FIG. 3A. Dox induction used concentrations that ranged from 0 ng/ml, 0.1 ng/ml, 1 ng/ml, 100 ng/ml, and 1000 ng/ml.

FIG. 3D shows mCherry and EGFP protein fluorescence at Day 4 as described in FIG. 3A with No Dox addition. EGFP is clearly visible as determined by the number and prevalence of green fluorescent cells.

FIG. 3E shows mCherry and EGFP protein fluorescence at Day 4 as described in FIG. 3A with addition of Dox at 100 ng/ml. mCherry is more visible as determined by increased number and prevalence of red fluorescent cells.

FIGS. 4A to 4D demonstrate the tunable switch of the master regulatory construct using Sleeping Beauty transposase.

FIG. 4A shows schematic diagrams of creating a tunable switch for the master regulatory construct. The gene encoding for Sleeping Beauty transposase (100×hsSB) is placed in reverse orientation in-frame to the TRE3G promoter while the gene encoding mApple fluorescent protein is placed in the forward orientation in-frame to the TRE3G promoter. A sequence encoding a Cre recombinase, reversible tetracycline transactivator (rtTA), and Blasticidin (Bsd) resistant genes are driven by CMV promoter (arrow). Addition of Dox results in inversion and excision events, resulting in loss of mApple and expression of 100×hsSB protein.

FIG. 4B demonstrates highly soluble Sleeping Beauty transposase (hsSB) mRNA induction in a dosage dependent manner by Dox. The hsSB mRNA was measured by RT-qPCR at Day 2 and Day 7 using cells that express the master regulatory construct as described in FIG. 4A. Varying levels of Dox induction was used ranging from 0 ng/ml, 10 ng/ml, and 100 ng/ml for 8 hrs at Day 0 and then removed from cell culture medium after 8 hrs. The hsSB expression is normalized to housekeeping gene, EIF3I and Day 0 timepoint. At Day 7, expression of hsSB mRNA is significantly reduced (dashed line, black dot) compared to Day 2 (solid line, gray dot).

FIG. 4C shows expression of rtTA mRNA measured at Day 2 post-Dox induction (0, 10, and 100 ng/ml) by RT-qPCR. As rtTA is driven by a ubiquitous promoter, the amount of rtTA mRNA expressed is not dependent on Dox concentration.

FIG. 4D shows expression of mApple mRNA measured at Day 2 post-Dox induction (0 and 10 ng/ml) by RT-qPCR. Following Dox induction (10 ng/ml), mApple mRNA expression is almost completely abrogated, demonstrating proof-of-concept of our tunable switch.

FIGS. 5A to 5 demonstrate the gene copy amplification of Sleeping Beauty transposon in a dosage-dependent manner.

FIG. 5A shows a SDS-PAGE reducing gel of purified hsSB transposase from E. coli, running at the expected size of 40 kDa.

FIG. 5B shows a spectrograph of Liquid chromatography-mass spectrometry (LC-MS) of purified hsSB from FIG. 5A with expected size at 40 kDa. The predominant peak occurs at 39.929 kDa and 3 minor peaks at 39.948, 40.028, and 40.107 kDa respectively.

FIG. 5C demonstrates gene copy amplification of transposon in a dosage-dependent manner of transposase. CHO-K1 cell line were transfected with 0, 2 μg, 10 μg or 50 μg of transposase and 30 μg of total plasmid DNA of the transposon expression constructs: SB-AR301 HC-Puro and SB-AR301 LC-Puro (black squares, solid lines) or NeonGreen control (gray triangles, dashed lines). Gene copy numbers were quantified by qPCR from genomic DNA at Day 3 post-transfection of Sleeping Beauty transposase (see Example 9).

FIG. 6 is a graph comparing the editing efficiency in a specific locus, Glu1 in CHO host cell line by easement of the percent indel (insertion/deletion). The Glul-specific sgRNA and Cas9 nuclease in CHO cells by T7 endonuclease 1 (T7E1) mismatch detection assay. The exon 4 and exon 7 of Glul were used as examples to demonstrate locus-specific targeting (Example 5). Editing at Glu1 exon 4 and Glu1 exon 7 was performed using ribonucleoprotein (RNP) containing Cas9 and a single guide RNA (sgRNA). The RNP complex was assembled in vitro. The sgRNA containing 20 bp target sequence of Glul exon 4 and exon 7 was used (SEQ ID NO: 4 and 6) Genomic DNA of edited CHO cells are isolated at 72 hours post-transfection and T7 endonuclease 1 (T7E1) mismatch detection assay used to routinely detect CRISPR-Cas9 mediated gene editing was used to measure % insertion/deletions (% Indel).

FIG. 7A is a schematic diagram of Zsgreen1 knock-in into an example SHL (NW_0036139321.1, 1,118,242-1,144,234 (+)) in CHO DG44 using the method described in FIG. 3. Zsgreen1 fluorescent protein expressed by CMV promoter is knocked into SHL (dotted) using ribonucleoprotein (RNP; black) complex containing Cas9+sgRNA. (SEQ ID NO: 17). Ribonucleoprotein (RNP3) complex consisting of Cas9 and sgRNA are assembled in vitro. Following RNP entry into the cell by electroporation, editing occurs via homology directed recombination (HDR) at the 5′ and 3′homology arm (HA). The Zsgreen1 is knocked into an example SHL (NW_003613932.1, 1,118,242-1,144,234 (+)) in CHO DG44. The image (FIG. 7B) represents successful knock-in characterized by single cell detection of fluorescent green protein expression. The image was collected using a single focal plane at 10× magnification.

### DETAILED DESCRIPTION

A number of methods and compositions are discussed in the Summary of the invention and further details are provided herein and in the Examples section. As would be readily appreciated by the skilled person, the disclosures can be read in combination.

The methods and systems described allow expression of polypeptide from one or more transposons in a eukaryotic host cell with a high level of expression control, while avoiding problems associated with random insertions in the cell genome. The systems employ a control construct and an expression construct. The control construct (master regulatory construct) includes a strictly controlled transposase sequence to provide control over the timing and extent of transpositions

These transposon systems are able to provide controlled non-random integration of transposons and inducible transposase into safe harbor locus (SHL). No delivery of transposase into the cell is needed because stable integration of transposase is achieved upon addition of antibiotic. This system allows one to fine tune the amount of transposase that is stably expressed in the cell in a tightly regulated temporal manner. Removal of antibiotic can be used to stop the amplification process so that no re-excision or re-integration can occur.

CHO production cell lines in biomanufacturing. Chinese hamster ovary (CHO) cells comprise a variety of lineages including CHO-DXB11, CHO-K1, CHO-DG44, and CHO-S. CHO-DG44 were generated by gamma irradiation to yield a cell line in which both alleles of the DHFR locus were completely eliminated. These DHFR-deficient strains require glycine, hypoxanthine, and thymidine for growth (Noguchi, 2012). Cell lines with mutated DHFR are useful for genetic manipulation as cells transfected with a gene of interest along with a functional copy of the DHFR gene can easily be screened for using thymidine-deficient media. Hence, CHO cells lacking DHFR are the most widely used CHO cells for industrial protein production.

Genetic engineering controls implemented by the presently disclosed invention enable the control of gene copy number, control of integration, targeted placement of the inserted gene within the genome. By combining the use of homology-directed repair (HDR) CRISPR/Cas9 together with AAV to deliver single stranded donor DNA (see text below), this improves on genetic stability and generates high titer mAb producing cell lines in a rapid manner. The use of adenoviral-associate virus (AAV) has advantages over traditional lentiviral approaches. As a method for gene insertion, it allows for vector design features that facilitate improved control over this process. However, AAV represents a viral vector with concerns regarding immunogenicity and insertional mutagenesis limiting its use for biomanufacturing.

Genetic stability is driven by control of site-specific integration. Productivity and stability are the two most crucial factors in stable cell line development. The traditional method to obtain high-expressing recombinant CHO cell lines for industrial production is random integration of the recombinant protein gene (Dorai, 2012). With the current technology, to obtain a clone with high expression levels, multiple rounds of screening by a selective marker are obligatory. Moreover, due to lack of control of insertion sites by random integration, protein productivity of some selected clones may diminish over time, causing instability of cell lines. Site-specific integration discussed herein further enables, e.g., copy number control, genetic stability, and stability of cell culture parameters. Alternative approaches using recombinase mediated cassette exchange (RMCE) have been employed to achieve site specific integration using Flp/FRT, Bxb1, Cre/loxP, and phiC31/R4 integrases. Typically, this kind of system requires the establishment of cell line platforms containing exogenous homologous recombination sites for target gene integration. The use of constitutively expressed Cre also has been demonstrated to have cytotoxic effect on mammalian cell lines, limiting it usage. Furthermore, these current systems do not allow for gene amplification or inducible gene expression systems, limiting their versatility in production cell lines.

Another system in use is mediated by engineered nucleases such as transcription activator like effector nucleases (TALENs), zinc finger nucleases (ZFNs), and clustered regularly interspaced short palindromic repeats (CRISPR)-associated (Cas) RNA guided nucleases. Compared to ZFNs and TALENs, CRISPR relies on ribonucleotide complex formation instead of protein/DNA recognition. The gRNAs can target nearly any sequence in the genome and they can be easily produced, thus making CRISPR more efficient and cost effective than both TALEN and ZFN (Gupta, 2014).

In contrast, our techniques combine, e.g., CRISPR/Cas9 and single gRNA for site-specific targeting, inducible and conditional site-specific recombinases, and three pairs of heterotypic recombination sites. These elements are arranged in unique polarized fashion to drive transposon mediated site-specific directed gene amplification to a selected, rather than random location, which is a key feature of this inventive approach enabled by our technology.

As described above, targeted insertions into the cell genome is preferred as it increases stability of transgene insertions. Our current approach achieves this by utilizing homology directed repair (HDR)-specific CRISPR/Cas9 to target transposons preferentially to SHL (see below). The advantage of this approach is that it enables efficient drug inducible gene amplification with control of copy number in addition to locus specific targeted insertion into a SHL in a rapid manner.

Transposable elements are a non-viral alternative for gene delivery. Introduction of desired transgenes in cells and organisms can use viral vectors which provide efficient gene transfer but require lengthy and expensive manufacturing for clinical use (Vargas, 2015). Viral-vector-encoded epitopes also bear a risk for inflammatory responses and preferential integration in transcribed regions may lead to adverse genomic changes. DNA transposons constitute a non-viral alternative for gene delivery. Expression manipulation using transposons typically requires, e.g., two essential components: the transposon DNA containing a genetic sequence of interest flanked by specific DNA end (e.g., homology arm HA) sequences and a transposase enzyme to insert the transposon sequence into a target (e.g., SHL) location.

DNA transposons are discrete pieces of DNA with the ability to change their positions within the genome via a “cut and paste” mechanism called transposition. Conventionally, both components are provided as plasmid DNA vectors and the transposase is expressed in the target cells. After expression, the transposase protein specifically binds the transposon ends of the cargo vector, excises the transgene and integrates it in the genome of the target cell (transposition). As transposons insert DNA self-sufficiently, they elicit similar transgenesis rates to gammaretroviral and lentiviral vectors. They have favorable attributes regarding immunogenicity, insertion profile, sequence capacity (up to 20-150 kb), complexity and cost for clinical use. Transposable elements can be viewed as natural DNA transfer vehicles that, similar to integrating viruses, are capable of efficient genomic insertion. In nature, these elements exist as single units containing the transposase gene flanked by inverted terminal repeats (ITRs) that carry transposase binding sites. However, under laboratory conditions, it is possible to use transposons as bi-component systems, in which virtually any DNA sequence of interest can be placed between the transposon ITRs and mobilized by trans-supplementing the transposase in the form of an expression plasmid or mRNA synthesized in vitro. In the transposition process, the transposase enzyme mediates the excision of the element from its donor plasmid, followed by reintegration of the transposon into a chromosomal locus. This feature makes transposons natural and easily controllable DNA delivery vehicles that can be used as tools for versatile applications.

The insertion pattern of most transposons remains unpredictable, although they have “hotspots” and “cold regions” on a genome-wide scale (Balasubramanian, 2016). Several approaches are commercially available, including the PiggyBac Transposon system (SBI System Biosciences), and Leap-In Transposase system (ATUM). Both approaches have been successfully used to rapidly generate stable pools with volumetric productivities higher than those produced by traditional gene amplification methods. However, one important shortcoming of current transposon systems, including Sleeping Beauty (SB), is that the transposition events that occur in target cells are poorly controlled, limiting its success in the use in production cell line of biomolecules. In particular, the use of transposase-encoding DNA causes extended protein expression and can even lead to transposase gene acquisition in target cells. This constitutes a need for improving both the control and safety of SB, which are also critical requirements for cell and gene therapy in general. As for all integrating vectors, transgene insertion by SB can cause insertional mutagenesis, activation of proto-oncogenes and genome rearrangements. This risk is proportional to the number of transgene integrations per genome and can be alleviated by controlled reduction of the number of integration events. The ability customizes and control for the number of inserted transgene copies per cell which has not been demonstrated for pre-existing transposon technologies, and as described in this inventive approach, is critical because optimal gene dosage is directly linked to the yield of the therapeutic product.

Modified versions of piggyBac and SB transposases have potentially wide-ranging applications, such as reversible transgenesis and modified targeting of insertions. Precise excision enables restoration of the donor site to its pre-transposon state. Mutational analysis of these transposases has identified specific sites that can modify excision and integration activity and include generation of excision competent/integration defective (Exc⁺Int⁻) transposases. This has the advantage of avoiding genome modification following either piggyBac or SB excision by limiting reintegration events. However, while these genome tools show promise, these mutants lack the ability to control for gene copy number, site integration, as well as targeted integration.

In summary, this lack of control over the timing and kinetics of transposase exposure bears the risk for ongoing and uncontrolled transposition, cytotoxicity and transgene remobilization, thus adding to concerns about the potential for adverse transformation of therapeutic cell products (Lee, 2015). Table 2 (above) summarizes the challenges faced by the current transposon technologies that include limited to no control over copy number amplification or drug inducibility for gene amplification. In contrast, the present invention overcomes these shortcomings by improved efficiency of transduction, the ability to control for copy number mediated by a drug-inducible method, site specific integration and ability to target the gene to a SHL.

Transposase Delivery Methods

Transposon-based systems are particularly promising for use in integrating a gene of interest into the genome, since they are considered to be less immunogenic and to have a much larger cargo capacity than viral vectors, while maintaining highly efficient transgene integration (Querques, 2019). The piggyBac transposon, which is derived from the cabbage looper moth Trichoplusia ni, is mobile in many different species, including human cells. Transposon vectors require two components: a plasmid DNA (pDNA) carrying the gene of interest, and a source of transposase. Usually, the source of transposase is a pDNA carrying the transposase cDNA under the control of a strong promoter. A principal drawback encountered using this strategy is the lasting presence of the transposase due to persistence of the episomal pDNA and/or the possible non-specific integration of the transposase gene in the genome. This could remobilize the transgene once it has been inserted, and thus lead to genotoxicity.

In an attempt to improve the biosafety of gene integration, the source of transposase is a messenger RNA (mRNA), instead of the commonly used pDNA. The advantages of mRNA delivery include the lability of mRNA, and that it is not integrated into the genome which eliminates the risk of long-lasting side effects. Use of mRNA is attractive when only high levels and/or short-term expression are required to achieve the desired effect. Consequently. mRNA is of particular relevance for engineering secure transposon systems with limited transposase expression. In this context, transgenesis protocols based on SB or piggyBac transposition providing the transposase in the form of mRNA are described for eukaryotic species and in cultured cell lines such as CHO (Galla, 2011).

Direct protein delivery of transposase have also been performed. There are drawbacks to these methods for transposase delivery to the cell. As the transposase can linger in the cell for days, the risk of transposon remobilization poses a huge concern especially for upstream and downstream processes during biomanufacturing.

There has been a need to develop an inducible method of stable and consistent transposase activation without the risk of remobilization events. This present system alleviates the limitations of the current delivery methods as it enables the creation of a bifunctional transposase dial that acts as both a molecular switch and amplifier whose amplitude can be controlled by different doses of antibiotic concentration. To do so, we combined several elements of inducible gene expression systems (see below).

Inducible Gene Expression Systems

Inducible gene expression systems (e.g. tetracycline and tamoxifen) can be used with site-specific recombinase (SSR) systems (Cre-loxP, Flp-FRT, and ΦC31) to control stage-specific transgenic expression in vivo (Olurinniji, 2016). In the Cre-loxP or Flp-FRT system, Cre or Flp recombinase recognizes the 34-bp nucleotide sequence named loxP or FRT and can precisely catalyze homologous exchange between the two loxP or the two FRT sites, respectively. No nucleotides are gained or lost in this process. In such exchanges, no additional elements, e.g., except for some monovalent or divalent cations, are needed. The Cre-loxP and Flp-FRT systems enable the specific manipulation of DNA based on the direction and location of the two loxPs or FRTs. Cre catalyzes the deletion of the DNA between the two loxPs when the two loxPs are in the same direction on one DNA molecule. When one loxP is on a linear DNA molecule and another loxP is on a circular DNA molecule, the circular DNA integrates into the linear DNA at the target. If two loxPs are in opposite directions, the fragment between them inverts. When one loxP is on a linear DNA and a second loxP is on another linear DNA, the two linear DNA molecules exchange a segment similar to chromosomal rearrangement. Similarly, the Flp recombinase requires a 48 bp Flp Recombination Target (FRT) sequence for recombination. Cells integrated with the FRT sequence are available commercially, with the Flp-In™ System, and have been used successfully to generate stable cell lines that consistently produce polyclonal antibodies. Current commercial technologies that utilize Cre-loxP or Flp-FRT are limited to ectopic expression or excision of single gene and there is limited success in mammalian production cell lines. Although powerful, the Cre-loxP and Flp-FRT systems remain imperfect. In theory, the recombination catalyzed by Cre or Flp is reversible. Occasionally, the equilibrium favors the undesired direction. For example, in site-specific insertion, the reaction is likely to cut out the already inserted site. Finding ways to control the reaction direction has become a big challenge for the Cre-loxP and Flp-FRT systems. Thus far, several methods have been developed, such as limiting the reaction time with a heat-shock promoter and introducing subtle mutations into the loxP or FRT sites to block the reverse reaction. To address the reverse reaction problem, a third SSR system, the ΦDC31 integrase system, was developed. The ΦDC31 integrase was derived from the Streptomyces phage ΦDC31 and catalyzes the recombination between the attP site (39 bp minimal size) and the attB site (34 bp minimal size), forming attL and attR sites. The ΦDC31 integrase cannot catalyze recombination between the attL and attR sites. Therefore, the ΦDC31 integrase can catalyze recombination only in a strictly controlled direction. However, several pseudo-ΦC31 integrase target (attP) sites have been found in human and mouse cells. If a vector with an attB site is transfected into mammalian cells, a high frequency of integration occurs at these pseudo-attP sites, thus limiting integration control.

The above methods have not been used to perform gene amplification in a site-specific manner. They have only been used to increase or delete gene products. To date, no existing approach allows for conditional activation of transposase for dosage-dependent gene amplification into SHL. The presently disclosed systems solve the many problems discussed above in unforeseen ways, e.g., by combining molecular biological features in unique complementary ways. For example, the controlled, stable, and tunable systems can utilize a unique combination of, e.g., heterotypic recombination Lox and Tet-On. Furthermore, site-specific gene amplification at SHL can be performed by combining CRISPR with inducible transposon technology.

We utilized a previously described doxycycline sensitive promoter (TRE3G) and Tet-On system (Gossen, 1992) to control the activation of Sleeping Beauty (SB) transposase (as shown in FIG. 2). In the Tet-On system (reverse tetracycline-controlled transactivator protein (rtTA) dependent), expression of the target gene is dependent on the activity of an inducible transcriptional activator. The transcriptional activator is regulated reversibly by the inducing ligand tetracycline or tetracycline derivatives such as doxycycline (Dox). Target gene expression is turned on by the inducing ligand. The transcription factor rtTA does not bind with tTA-responsive promoter (TRE3G) without tetracycline or Dox; hence, the gene under study is not expressed. After Dox is added, it binds with rtTA, the Dox-rtTA complex binds with TRE3G, and target gene expression is initiated. Similar functional activator/promoter combinations are envisioned.

A preferred embodiment of the invention is the stable expression of transposase switch in the cell. The transposase is engineered to switch from “off” to “on” state. For example, this can be accomplished by positioning a required element, such as a transposase sequence in a non-functional reverse orientation, flanked by two heterotypic lox sites in opposite orientations. In such a case, Cre activity can invert or switch the sequence to a functional forward orientation through a two-step inversion and excision event. For example, as shown in FIGS. 2, introduction of Dox can result in inversion of the SB sequence to allow functional expression. The transposase (e.g., SB) can thus be switched from an “off” to “on” state upon introduction of doxycycline, importantly allowing the level of transposase activation depending on the concentration of the Dox. This control is reversible by withdrawing the Dox.

Amplifying Gene Copy Number of a Gene of Interest Using an Inducible Transposon System

A method of gene amplification uses an inducible transposon system comprising of the following constructs:

A master regulatory construct containing inducible transposase cassette containing the following elements (FIG. 1B):
1) A transposase in reverse orientation wherein a TRE3G promoter is located upstream of a transposase;
2) The gene encoding for Cre, Flp or combinations of Cre and Flp recombinase under the control of TRE3G promoter;
3) The gene encoding for reversible tetracycline transactivator (rtTA) under the control of a EF1A promoter;
4) A heterotypic lox or FRT recombination site placed in forward orientation downstream of TRE3G promoter and upstream of transposase; and,
5) A heterotypic lox or FRT recombination sites placed in reverse orientation downstream of reverse orientated transposase.

A transposon expression construct containing the following elements (FIG. 1C):
1) 5′ inverted terminal repeats (ITRs) unique to transposon
2) 3′ inverted terminal repeats (ITRs) unique to transposon
3) Dedicated promoters upstream of the genes to be amplified comprising of one or more of the following: CAG, SV40, hPGK, UBC, EF1a, CMV, and Cbh.
4) The coding gene(s) to be amplified flanked by 5′ and 3′ inverted terminal repeats (ITRs) of a transposase.

In a preferred embodiment, the invention provides a molecular method to induce transposase activation using constructs comprising the following elements (FIG. 1B):
A gene encoding for a Transposase in reverse orientation;
A modular combination of 2 sets of heterotypic lox or FRT homologous recombination sites in opposite orientation under the control of Tetracycline-response elements third generation (TRE3G) promoter, and,
The use of recombinase that recognizes heterotypic lox or FRT whose gene expression is under the control of TRE3G.

In a further preferred embodiment, the system according to the invention includes control of gene copy numbers of gene(s) of interest. The gene copy number amplification is controlled by concentration of doxycycline.

In another preferred embodiment, the system includes control of the gene integration sites to safe harbor loci (SHL) through the use of CRISPR enzymes.

In a preferred embodiment, the invention refers to a method constructing a high mAb producing CHO, HEK293T, SP2, Sp/0, Vero or NS0 cell line by amplifying gene copy number of endogenous H&L genes using an inducible transposon system. All descriptions are the same as described above, but for heavy and light chain genes of the respective monoclonal antibody:
1) The transposon expression construct containing heavy and light chain genes of monoclonal antibodies is flanked by transposase inverted terminal repeats (ITRs) upstream of the gene cassette containing heavy and light chain genes (FIG. 1C).
2) The gene cassette containing heavy and light chain genes comprise of the following (FIG. 1C):
   the first ubiquitous promoter is used to drive expression of light chain genes; wherein the promoters used is one of the following: CAG, SV40, hPGK, UBC, EF1a, CMV, and Cbh.
   a second ubiquitous promoter is used to drive expression of heavy chain genes; wherein the promoters used is one of the following: CAG, SV40, hPGK, UBC, EF1a, CMV, and Cbh.

In a preferred embodiment, the invention refers to a method of constructing a high mAb producing CHO, HEK293T, SP2, Sp/0, Vero or NS0 cell line by amplifying gene copy number of endogenous H&L genes using an inducible Sleeping Beauty transposon system.

All descriptions are the same as above but includes stable cell line selection comprising the following steps:
1) Cassette generation in mammalian expression vector, lentivirus (LV) transfer vector, or adenovirus associated (AAV) vector,
2) Packaging of LV or AAV into 293FT;
3) Purification of LV or AAV;
4) Titer quantification of LV or AAV;
5) Transfection or transduction of cassettes into CHO, HEK293T, SP2, Sp/0, Vero or NS0 cell lines; and,
6) Stable selection of CHO, Sp2/0 or NS0 cells containing stable integration of amplified heavy and light chain genes using nanowells.

In a more preferred embodiment, the invention refers to a method constructing a high mAb producing CHO, SP2 or NS0 cell lines by amplifying gene copy number of endogenous HC & LC genes using an inducible Sleeping Beauty transposon system as described above and preferentially using CAG promoter for expression of mAb heavy chain genes and EF1A promoter for expression of light chain genes.

In a more preferred embodiment, the invention refers to a method for amplifying and controlling gene copy number of endogenous HC &LC genes using specific concentrations of drug (see below):
1) Monoclonal Ab (mAb) HC+LC gene copy amplification by the addition of Doxycycline concentrations in the range of 0.1-1000 ng/ml for 2-72 hours;
2) Gene copy amplification of transposon quantified by quantitative real-time PCR

In a preferred embodiment, the invention refers to method of targeted insertion by knock-in into a CHO safe harbor locus (SHL) using CRISPR and AAV defined by Table 3, below. Targeted insertion in host cell line has been demonstrated to improve stability of transgenes and recombinant proteins and antibodies. Any of the CRISPR enzymes can be employed to place immunoglobulin (Ig) gene sequences in the targeted location within the cell genome of either the human cell line or the CHO cell line to enable CRISPR mediated activation of the desired transcription factors. The method specifies placement of the Ig gene sequences in an area within the genome that has minimal impact on other cellular functions (safe harbor locus). The locus-specific targeting of the Ig gene sequences is achieved using an adeno-associated virus (AAV) which has the advantage of enabling site-specific placement with improved control and productivity compared to random placement.

In a preferred embodiment, the invention refers to targeted insertions of human HC &LC chain genes in CHO chromosomal regions specified in Table 3. Transposon expression constructs as described above were delivered with Cas9 RNPs and AAV2 into candidates of safe harbor locus.

TABLE 3
Exemplary CHO safe harbor loci (SHL)

| | Chromosomal | | |
|---|---|---|---|
| | Region Seq ID | | |
| Chromosomal Region | No. | Gene | gRNA Seq ID NO |
| NW_003614386.1 | SEQ ID NO: 78 | Bmp5 | SEQ ID NO: 12-16 |
| 59948-293,109 (+) | | | |
| NW_003614117.1 | SEQ ID NO: 79 | non-coding, | SEQ ID NO: 24-26 |
| 132,089-1,168,208 (+) | | unannotated | |
| NW_003614502.1 | SEQ ID NO: 80 | non-coding, | SEQ ID NO: 27-29 |
| 234,676-514,621 (+) | | unannotated | |
| NW_003613746.1 | SEQ ID NO: 81 | non-coding, | SEQ ID NO: 30-32 |
| 1,864,326-2358326 (+) | | unannotated | |
| NW_003614999.1 | SEQ ID NO: 82 | Ssbp2 | SEQ ID NO: 33-35 |
| 207,362-379,237 (−) | | | |
| NW_003613587.1 | SEQ ID NO: 83 | Trmt6 | SEQ ID NO: 36-38 |
| 5,610,130-5,622,911 (+) | | | |
| NW_003614241.1 | SEQ ID NO; 84 | non-coding, | SEQ ID NO: 39-41 |
| 177,611-966,669 (+) | | unannotated | |
| NW_003615469.1 | SEQ ID NO: 85 | Clcc1 | SEQ ID NO: 42-44 |
| 84,472-104,058 (−) | | | |
| NW_003617368.1 | SEQ ID NO: 86 | Fam114a1 | SEQ ID NO: 45-47 |
| 4,201-47,792 (+) | | | |
| NW_003614039.1 | SEQ ID NO: 87 | Lrba | SEQ ID NO: 48-50 |
| 477,612-1,037,522 (+) | | | |
| NW_003613853.1 | SEQ ID NO: 88 | non-coding, | SEQ ID NO: 51-53 |
| 898,066-1,849,349 (+) | | unannotated | |
| NW_003614008.1 | SEQ ID NO: 89 | Dcn | SEQ ID NO: 54-56 |
| 416,333-453,059 (−) | | | |
| NW_003613824.1 | SEQ ID NO: 90 | Cep128 | SEQ ID NO: 57-59 |
| 918,001-1,253,950 (−) | | | |
| NW_003614624.1 | SEQ ID NO: 91 | Aacs | SEQ ID NO: 60-62 |
| 98,124-141,718 (+) | | | |
| NW_003613881.1 | SEQ ID NO: 92 | AldH5A1 | SEQ ID NO: 63-65 |
| 1,561,114-1,583,851 (−) | | | |
| NW_003613796.1 | SEQ ID NO: 93 | Smad6 | SEQ ID NO: 66-68 |
| 180,595-249,548 (+) | | | |
| NW_003613718.1 | SEQ ID NO: 94 | non-coding, | SEQ ID NO: 69-71 |
| 2,206,187-2,528,069 | | unannotated | |
| NW_003615261.1 | SEQ ID NO: 95 | non-coding, | SEQ ID NO: 72-74 |
| 117,306-308,958 | | unannotated | |
| NW_003613932.1 | SEQ ID NO: 96 | Hprt1 | SEQ ID NO: 17-23 |
| 1,118,242-1,144,234 (+) | | | |
| NW_003613637.1 | SEQ ID NO: 97 | Putative Rosa26 | SEQ ID NO: 75-77 |
| 132,002 to 132,401 (+) | | | |
| NW_003613921.1 | SEQ ID NO: 98 | Glul | SEQ ID NO: 1-7 |
| 1,427,960-1,435,423 (+) | | | |
| NW_003615627.1 | SEQ ID NO: 99 | Kiaa1551 | SEQ ID NO: 8-11 |
| 31,545-56,663 (+) | | | |

Methods can include the steps of:
1) Inserting transposon expression construct or cassette of heavy and light chain antibody genes or CDR regions into AAV2 donor delivery vector or ssDNA delivery.
2) Packaging and purification of AAV2 virus to deliver ssDNA into CHO host cell line; wherein AAV vector insert encodes for transposon described in 1) flanked by 50-750 bp homology domain for targeted knock-in of transposon; and, AAV2 virus is packaged in HEK 293T cells expressing simian virus large (SV40) large antigen.
3) Targeted dual site knock-in by methods of preparation of pre-assembled ribonucleoprotein (RNP) and AAV2 virus:
i. Wherein pre-assembled RNP uses recombinant CRISPR enzyme
ii. Wherein CRISPR enzyme comprises of Cas9. Cas12a (Cpf1), Cas12b, Cas13, Cas14, CasX or CasY.
4) Selection of recombinant CHO cell line with amplified gene by e.g. nanoculture and preferably on a single cell level.

In a preferred embodiment, the methods above were used to generate a targeted insertion knock-in of inducible transposase into a CHO safe harbor locus (SHL). In one preferred embodiment, the invention refers to a method of generating targeted insertion knock-in of transposon expression construct containing a gene of interest into a CHO safe harbor locus (SHL). In a more preferred embodiment, the invention refers to a method of generating targeted insertion knock-in of transposon expression constructs containing antibody heavy and light chain genes into a CHO safe harbor locus (SHL).

In an embodiment, the invention refers to a method constructing a high productivity monoclonal antibody mammalian production cell line using the inducible Sleeping Beauty transposon system for amplification of heavy and light chain genes wherein the master transcription regulatory elements (MTRE) of said production cell line are activated.

In a further embodiment, the invention refers to a method constructing a high productivity monoclonal antibody mammalian production cell line using an inducible Sleeping Beauty transposon system for amplification of heavy and light chain genes targeted to SHL, wherein the MTRE of said production cell line are activated.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

### Example 1: Generation of Master Regulatory Construct Expressing Inducible Transposase, ISB-Bsd (FIG. 1B, FIG. 2)

Four separate cassettes were generated by de novo synthesis followed by subcloning into high copy cloning vector, pUC19: TRE3G-hsSB(rev), gene encoding for Cre recombinase, Blasticidin resistance (Bsd) and rtTA genes. DNA containing TRE3G-hsSB (rev) cassette containing the TRE3G promoter, a highly soluble Sleeping Beauty (hsSB) transposase flanked by loxP and loxN in forward orientation at the 5′ end and loxP and loxN at the reverse orientation at the 3′ end (as shown in FIG. 1B, FIG. 2) were synthesized de novo and subcloned into a high-copy cloning vector with pUC19 backbone at 5′ BsiWI and 3′ BsrGI cloning sites. CMV-Cre, CMV-rtTA and CMV-Bsd were generated by subcloning Cre, rtTA and Bsd downstream of CMV promoter using In-Fusion cloning (Takara Bio). Individual fragments were assembled by Gibson assembly and verified by Sanger sequencing (Sanger, 1975). The final master regulatory construct was subcloned into AAV transfer vector (Takara Bio) by In-Fusion cloning and labeled as iSB-Bsd.

### Example 2: Generation and Characterization of Inducible Master Regulatory Construct Expressing Fluorescent Reporters (FIG. 3)

TRE3G-EGFP(fw)-mCherry(rev) was generated using strategy as described for TRE3G-hsSB in Example 1 with the following modifications: individual DNA fragments containing the TRE3G promoter, and fluorescence level of acquired images were quantified by Zen analysis software. As shown in FIG. 3C-3E, addition of Dox induces expression of mCherry in a dosage dependent level on both the RNA level (FIG. 3B) and protein level (FIG. 3C-E).

### Example 3: Generation and Characterization of Master Regulatory Construct Expressing Inducible Transposase with mApple Reporter (FIG. 4A)

TRE3G-mApple(fw) hsSB(rev) was generated using strategy as described for TRE3G-hsSB in Example 2 with the following modifications: individual DNA fragments containing the TRE3G promoter, a gene encoding for mApple (in forward orientation) and hsSB (in reverse orientation) flanked by loxP and loxN in forward orientation at the 5′ end and loxP and loxN at the reverse orientation at the 3′ end (see FIG. 4A) were synthesized de novo and subcloned into a high-copy cloning vector with pUC19 backbone at 5′ BsiWI and 3′ BsrGI cloning sites. Doxycycline (Dox) was added to cell line expression construct as described in FIG. 4A at 10 and 100 ng/ml for 8 h in serum-free chemically defined EX-Cell CHO Fed-Batch medium (Sigma Aldrich) and removed following 8 h post-induction. Total RNA at Day 0, Day 2, and Day 7 was purified by Quick RNA Kit (Zymo Research) and reverse transcription quantitative polymerase chain reaction (RT-qPCR) for EIF3I, mApple, rtTA and hsSB was performed using TB Green fluorescent dye (Takara Bio). EIF3I is used as a housekeeping gene and RNA levels were normalized to EIF3I and Day 0 timepoint. As shown in FIG. 4B, hsSB is induced in a concentration dependent manner with strongest expression at Day 2. Following Dox addition, rtTA mRNA is largely unaffected (FIG. 4C) while mApple is significantly reduced (FIG. 4D).

### Example 4: Transposon Expression Construct SB-AR301 HC-Puro and SB-AR301 LC-Puro

To generate SB-AR301 HC-Puro and SB-AR301 LC-Puro, we generated SB-AR3201 HC, SB-AR301 LC, and mPGK-Puro constructs and subcloned into a high-copy cloning vector as described above. Heavy and light chain genes of AR301 mAb (AR301 HC and LC, respectively) were generated by de novo synthesis. The final transposon expression construct was assembled by Gibson assembly and subcloned into pUC19 cloning vector containing Ampicillin resistant gene (New England Biolabs, NEB).

### Example 5: Design, Generation and Validation of sgRNAs and HDR Constructs for Targeted Integration into SHL Using CRISPR/Cas

For each chromosomal region defined as CHO safe harbor locus as outlined in Table 3, single guide RNAs (sgRNAs) were designed to target a stretch of 20 nucleotides (nt) of the genomic region specified in Table 3 (SEQ ID NO: 1-77) containing Cas9 protospacer-associated motif (PAM). The sgRNA target sequence was designed from Cricetulus griseus assembly (GenBank accession numbers: GCA_003668045.1, GCA_000419365.1, GCA_000223135.1 and GCA_000448345.1) using Benchling (https://benchling.com) or CHOP CHOP (https://chopchop.cbu.uib.no/). The sgRNAs were synthesized in vitro (Synthego). DNA sequences of chromosomal regions specified in Table 3 are listed in SEQ ID NO: 78-99.

For validation of sgRNAs, T7 endonuclease 1 (T7E1) mismatch detection assay was used. T7E1 detection assay has been the gold standard to quantify editing efficiency. T7E1's functional role is to detect structural deformities in heterodupliexed DNA. Following electroporation, genomic DNA surrounding the specified locus is amplified by PCR. The resulting PCR product is denatured and re-annealed by slow cooling. If an erroneous NHEJ event occurs, as is the case of Cas9 editing, heteroduplex will form between amplicons of different lengths (mutant and WT amplicons), resulting in structural DNA changes that can be recognized and cleaved by T7E1. Such banding patterns of cleaved produces are compared to control and experimental samples (Mashal et al., 1995). We used the locus of Glutamine synthetase (Glul), designing sgRNA to target exon 4 and exon 7 (FIG. 6; SEQ ID NO: 4, 6). Cas9 ribonucleoprotein (RNP) and sgRNA were assembled in vitro wherein each sgRNA contains 20 bp target sequence of Glul exon 4 and exon 7. Genomic DNA of edited CHO cells are isolated at 72 hours post-transfection and T7 endonuclease 1 (T7E1) mismatch detection assay used to routinely detect CRISPR-Cas9 mediated gene editing was used to measure % insertion/deletions (% indel; FIG. 6). As shown in FIG. 6, targeting specific at exon 7 and exon 4 of Glul gene results in 32% and 38% editing, respectively while no editing was observed in no sgRNA control.

### Example 6: Generation of HDR Constructs for Targeted Integration into SHL Using CRISPR/Cas

Generation of pCMV-HDR-ZsGreen1-AAV
AAV homology directed repair (HDR) donor cassette containing CMV-ZsGreen1 flanked by 750 bp 5′ and 3′ homology arms targeting genomes regions as presented in Table 3 were subcloned into pCMV-AAV (Takara Bio) at the EcoRI (5′) and BamHI (3′) cloning sites using In-Fusion cloning (Takara Bio) and labeled as pCMV-SHL HDR-ZsGreen1-AAV (as described above). The 5′ and 3′ homology arms for each locus were synthesized de novo and subcloned into pUC19 cloning vector as described above.

### Example 7: Packaging, Purification, and Titering of AAV2-SHL HDR-Zsgreen1 Particles

AAV2 serotype was packaged using AAVpro Helper free system (Takara Bio) in 293FT cells containing large SV40 antigen (Invitrogen). 293FT cells were grown in media recommended per manufacturer's instructions. At 1 day prior to transfection, 293FT cells were re-seeded to achieve 70-80% confluency the next day without the use of antibiotics. A total of 28 μg of DNA of pHelper-AAV, pRC2-mi342, and pCMV-SHL HDR-Zsgreen1-AAV were transfected using Xfect Polymer (Takara Bio, FIG. 4).

After 72 hours of transfection, the cell pellet containing AAV2 serotype were extracted and purified using AAVpro extraction solution (Takara Bio). Virus titer (in viral genome/ml or vg/ml) was determined by real-time quantitative PCR (qPCR) using TB Green EX Taq II following DNase I treatment for 15 min at 37° C., DNase I inactivation at 95° C. for 10 min and lysis at 70° C. for 10 min.

### Example 8: Transfection/Transduction into Mammalian Cell Lines

Cas9 or Cpf1 ribonucleoprotein (RNP) complex were assembled in vitro using 1:2 molar ratio of Cas9 or Cpf1 containing nuclear localization sequence, sgRNA containing 20-nt target sequence and scaffold; 5 μM of Cas9 or Cpf1 was used. Following RNP assembly at 37° C. for 5 min, 4.8×10⁵ viable cells of serum-free adapted CHO cells such as CHO DG44, CHO-K1 or CHO-S (Invitrogen) were electroporated using Neon transfection system with a voltage of 1700, pulse width of 10 ms, and 3 pulses were applied to cells containing RNP complex. Following electroporation, cells were transduced with AAV2-SHL HDR at 5,500 vg/cell.

### Example 9: Control of Gene Copy Number of Interest (FIG. 5C)

Gene copy number of transpositions of heavy and light chain gene of monoclonal antibody (mAb) against AR-301 or NeonGreen were determined by real-time quantitative PCR (qPCR) using KiCqStart® Probe qPCR Master Mix (Qiagen). TB Green Ex Taq II Polymerase. 6.25×10⁶ viable CHO-K1 cells were electroporated with electroporation buffer containing 10 μg of DNA of SB-AR301 HC-Puro and 20 μg of DNA of SB-AR301 LC-Puro or 30 μg of DNA of SB-NeonGreen cassette with a voltage of 1700, pulse width of 10 ms; 3 pulses were applied to cells. After 4 hours post-transfection, varying amounts of purified SB transposase was transfected into CHO-K1: 0, 2 μg, 10 μg or 50 μg with Neon electroporation system with a voltage of 1700, pulse width of 10 ms; 3 pulses and cultured for 3 days following transpositions.

Genomic DNA from cells after 3 days following transpositions were isolated using Quick-DNA Microprep Plus Kit (Zymo Research). Following gDNA isolation, qPCR was done using probes specific to left end of SB transposon and single-copy CHO-K1 β-Actin genes. The program used for thermal cycling conditions was as follows: 95° C. for 30 sec; 40 cycles of 95° C. for 5 sec, 60° C. for 30 sec. Copy numbers were determined by normalizing to single copy β-Actin CHO reference genes (NM_007393).

### Example 10: Control of Gene Integration Sites to SHL

Multiplex arrays containing single, double or triple modules (Multi-5×SHL, Multi-10×SHL, and Multi-15×SHL) of RNA Pol III U6 promoter driving crRNA array of 19 nt target sequence, five spacers separated by direct repeats (DRs) from CRISPR locus of Acidaminococcus (AsCpf1), and crRNA scaffold 4n96 are synthesized de novo and in vitro transcribed (Teng et al., 2019).

His-tagged Cpf1-SBcat was generated by fusion of domains of Cpf1: WEDI-III, alpha-helical recognition lobe (REC1-2), and P1 domains to the catalytic domain of Sleeping Beauty transposase (aa 112-340). DNA sequence coding for Cpf1-SBcat was subcloned in pET-21a-d(+) (Novagen), expressed and purified by Ni-NTA Agarose column. His-tag was cleaved off by Thrombin cleavage kit (Sigma).

Cpf1 or Cpf1-SBcat RNP is assembled as described: 5 μM of Cpf1 or 5-20 μM Cpf1-SBcat is added to crRNA array (molar ratio of AsCpf1 or Cpf1-SBcat to crRNA is 1 to 2). Following in vitro assembly of RNP, Dox-treated CHO-iSB or CHO DG44 with were electroporated with electroporation buffer containing Cpf1 or Cpf1-SBcat RNP with 1 μg, 5 μg, or 20 μg of DNA of SB-AR301 HC+LC Puro with a voltage of 1700, pulse width of 10 ms; 3 pulses were applied to cells.

Dox-treated CHO-iSB transfected with SB-AR301 HC-Puro and SB-AR301 LC-Puro were transduced with AAV2 as described in Example 6 to deliver ssDNA: AAV2-SHL HDR into SHL. Single cell clones were selected on nanowells by IgG diffusion assay and grown in ClonaCell CHO animal-component free (ACF) supplemented with ClonaCell™-CHO ACF supplement (Stem Cell Technology), 1% Hypoxanthine Thymidine (HT), 1% Penicillin Streptomycin and Puromycin (6.25 μg/ml).

Single cell clones with CHO DG44 transfected with SB-AR201 HC+LC Puro were selected on nanowells by IgG diffusion assay and grown in ClonaCell CHO animal-component free (ACF) supplemented with ClonaCell™-CHO ACF supplement (Stem Cell Technology), 1% Hypoxanthine Thymidine (HT), 1% Penicillin Streptomycin and Puromycin (6.25 μg/ml).

For sequence verification of CHO cell line with SB-mediated transposition, 1 μg genomic DNA was purified and PCR amplicon libraries are generated by two-step PCR protocol using Illumina sequencer. Equimolar amounts of purified, individually barcodes libraries were pooled and sequenced bidirectionally with a read length of 250 bp. Sequences were aligned with CHO reference genome and mapped.

### Example 11: Manufacturing with Standard and Modified Cell Lines

10-day batch productivity in shake flasks using seeding density of 2E5 viable cells/ml was performed in 125-ml shake flask in Ex-Cell CD Fusion medium with 6 mM L-Glutamine. The specific growth rate, p is calculated based on equation 1 (Eq. 1).

$$μ = \frac{{lnX}_{V 1} - {lnX}_{V 0}}{{t}_{1} - {t}_{0}} \qquad Eq . 1$$

where X_{V1} and X_{V0} are the viable cell densities. Cell counts are determined by adding equal volume of cell suspension to 0.4% Trypan Blue and counted using BioRad TC. Sandwich immunoglobulin G (IgG) enzyme-linked immunosorbent assay (ELISA) was performed to determine the monoclonal antibody (mAb) concentration secreted by CHO cell lines. The cell specific productivity (qP) is calculated using equation 2 (Eq. 2):

$$q P = \frac{Δ P}{{\int }_{0}^{t} Ndt} \qquad Eq . 2$$

### Example 12: 60-Generation Clonal Stability

The doubling time of cell lines is determined based on equation 3. (Eq. 3):

$${T}_{d} = ( {t}_{1} - {t}_{0} ) * \frac{ln ( 2 )}{ln ( \frac{{X}_{V 1}}{{X}_{V 0}} )} \qquad Eq . 3$$

Cell lines are propagated using seeding density 2-3×10⁵ viable cells/ml in shake flask using standard cell culture procedures. Every 8-10 generations, 10-day stability studies are performed by seeding cell lines at 2E5 viable cells/ml in 20 ml seeding volume in shake flasks at 110 rotations per minute (rpm). Endpoint titer at Day 10 is determined by ELISA.

### REFERENCES

1. Balasubramanian, S., Rajendra, Y., Baldi, L., Hacker, D. L., & Wurm, F. M. (2016). Comparison of three transposons for the generation of highly productive recombinant CHO cell pools and cell lines. Biotechnology and Bioengineering, 113(6), 1234-1243. doi: 10.1002/bit.25888.
2. Bobo, B., Phelan, D., Rebhahn, J., Piepenbrink, M. S., Zheng, B., Mosmann, T. R., DeLouise, L. A. (2014). Microbubble array diffusion assay for the detection of cell secreted factors. Lab on a chip, 14(18), 3640-3650. doi:10.1039/c41c00580e
3. Canny, M. D., Moatti, N., Wan, L., Fradet-Turcotte, A., Krasner, D., Mateos-Gomez, P. A., Durocher, D. (2018). Inhibition of 53BP1 favors homology-dependent DNA repair and increases CRISPR-Cas9 genome-editing efficiency. Nature biotechnology, 36(1), 95-102. doi:10.1038/nbt.4021
4. Dorai, H., Corisdeo, S., Ellis, D., Kinney, C., Chomo, M., Hawley□Nelson, P., Moore, G., Betenbaugh, M. J. and Ganguly, S. (2012). Early prediction of instability of chinese hamster ovary cell lines expressing recombinant antibodies and antibody□ fusion proteins. Biotechnol. Bioeng., 109: 1016-1030. doi:10.1002/bit.24367
5. Fischer S, Handrick R, Otte K. (2015). The art of CHO cell engineering: A comprehensive retrospect and future perspectives. Biotechnol Adv. 33:1878-1896. doi: 10.1016/j.biotechadv.2015.10.015.
6. Galla, M. et al. (2011). Avoiding cytotoxicity of transposases by dose-controlled mRNA delivery. Nucleic Acids Res. 39, 7147-7160.
7. Gibson, D., Young, L., Chuang, R. et al. (2009). Enzymatic assembly of DNA molecules up to several hundred kilobases. Nat Methods 6, 343-345 doi:10.1038/nmeth.1318
8. Gossen M, Bujard H. (1992). Tight control of gene expression in mammalian cells by tetracycline-responsive promoters. PNAS. 89(12):5547-5551. doi: 10.1073/pnas.89.12.5547.
9. Gupta, R. M., & Musunuru, K. (2014). Expanding the genetic editing tool kit: ZFNs, TALENs, and CRISPR-Cas9. The Journal of Clinical Investigation, 124(10), 4154-4161. doi:10.1172/JC172992
10. Lee J S, Kallehauge T B, Pedersen L E, Kildegaard H F (2015). Site-specific integration in CHO cells mediated by CRISPR/Cas9 and homology directed DNA repair pathway. Sci Rep 5:8572. https://doi.org/10. 1038/srep08572
11. Noguchi C, Araki Y, Miki D, Shimizu N (2012). Fusion of the Dhfr/Mtx and IR/MAR gene amplification methods produces a rapid and efficient method for stable recombinant protein production. PLoS ONE 7(12): e52990. https://doi.org/10.1371/journal.pone.0052990
12. Olorunniji, F. J., Rosser, S. J. & Stark, W. M. (2016). Site-specific recombinases: molecular machines for the genetic revolution. Biochem J 473, 673-684, https://doi.org/10.1042/BJ20151112
13. Querques, I., Mades, A., Zuliani, C. et al. (2019). A highly soluble Sleeping Beauty transposase improves control of gene insertion. Nat Biotechnol 37, 1502-1512 doi:10.1038/s41587-019-0291-z
14. Sanger F, Coulson A. R, (1975). A rapid method for determining sequences in DNA by primed synthesis with DNA polymerase. J. Mol. Biol. 94 (3): 441-8. doi:10.1016/0022-2836(75)90213-2. PMID 1100841.
15. Saunders, F., Sweeney, B., Antoniou, M. N., Stephens, P., & Cain, K. (2015). Chromatin function modifying elements in an industrial antibody production platform-comparison of UCOE, MAR, STAR and cHS4 elements. PLoS One, 10(4), e0120096. doi: 10.1371/journal.pone.0120096.
16. Teng F, Li J, Cui T, et al. (2019). Enhanced mammalian genome editing by new Cas12a orthologs with optimized crRNA scaffolds. Genome Biol. 20(1):15. doi:10.1186/s13059-019-1620-8
17. Vargas, J. E. et al. (2016). Retroviral vectors and transposons for stable gene therapy: advances, current challenges and perspectives. J. Transl. Med. 14, 288.
18. Yoshida, J., Akagi, K., Misawa, R. et al. (2017). Chromatin states shape insertion profiles of the piggyBac, Tol2 and Sleeping Beauty transposons and murine leukemia virus. Sci Rep 7, 43613.doi:10.1038/srep43613.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

Incorporated by reference into the present specification is the sequence listing filed as 136-001310US_Seq_List.xml, created Nov. 14, 2022, of 157,646 bytes.

What is claimed is:

1. A system for conditional control of gene copy number amplification in an expression host cell, the system comprising:
1) a nucleic acid master regulatory construct comprising:
a transposase sequence in reverse orientation downstream from a promoter sequence, which promoter sequence is controlled by an inducing agent;
a Cre recombinase sequence with expression controlled by the inducing agent; and,
wherein the transposase sequence is upstream from a reverse oriented heterotypic first Lox sequence and downstream from a second Lox sequence, which second lox sequence is forward oriented between the promoter sequence and the transposase sequence; and,
wherein the stop sequence is bracketed by a pair of forward oriented lox sequences;
2) an expression transposon nucleic acid construct comprising:
a transposon sequence encoding a protein sequence of interest flanked by inverted terminal repeats (ITRs), which ITRs are the binding target of the transposase; and,
a binding site (BS) for the first transcriptional activator upstream from the ITRs;
wherein the presence of the inducing agent induces expression of Cre, resulting in CRE/Lox inversion of the transposase to forward orientation and resulting in excision of the stop sequence, thus resulting in expression of the transposase; enabling insertion of the transposon at particular safe harbor loci (SHL) of interest for expression of the protein of interest.

2. The system of claim 1, wherein the transposase is a Sleeping Beauty transposase.

3. The system of claim 1, further comprising one or more second transcriptional activators in the form of a ligand/receptor inducing agent.

4. The system of claim 3, wherein one or more of the master regulatory control construct promoter sequences is a TRE3G promoter and the inducing agent is doxycycline/reversible tetracycline transcriptional activator (Dox/rtTA).

5. The system of claim 1, wherein the protein of interest is an antibody heavy chain or an antibody light chain.

6. The system of claim 1, wherein the host cell is selected from the group consisting of: SP2/0, NS0, HEK293T, Vero, and CHO.

7. The system of claim 1, wherein insertion of the transposon is enabled by knock-in into one or more Safe Harbor Loci (SHL) by AAV and CRISPR enzymes Cas9, Cas12a (Cpf1), Cas12b, CasX, or CasY.

8. The system of claim 7, wherein the SHL is selected from the SHLs of Table 3.

9. The system of claim 1, wherein the master regulatory construct and expression transposon construct are on the same nucleic acid.

10. The system of claim 1, wherein the master regulatory construct further comprises a ubiquitous promoter controlling expression of the reversible tetracycline transcriptional activator (rtTA) and a selective pressure resistance factor (e.g., Bsd).

11. The system of claim 10, wherein the ubiquitous promoter is selected from the group consisting of: EF1a, CAG, Cbh, SV40, UBC, CMV, EFS, and CMV promoter combined with CMV immediate early enhancer elements.

12. A nucleic acid master regulatory construct comprising:
a transposase sequence in reverse orientation downstream from a promoter sequence, which promoter sequence is controlled by an inducing agent;
a Cre recombinase sequence with expression controlled by the inducing agent;
wherein the transposase sequence is upstream from a reverse oriented first heterotypic Lox sequence and downstream from a second Lox sequence, which second lox sequence is forward oriented between the promoter sequence and the transposase sequence; and,
wherein the stop sequence is bracketed by a pair of forward oriented lox sequences
whereby the presence of the inducing agent induces expression of Cre, resulting in CRE/Lox inversion of the transposase to forward orientation and resulting in excision of the stop sequence, thus resulting in expression of the transposase.

13. A method for conditional control of gene copy number amplification in an expression host cell, the method comprising:
1) providing a nucleic acid master regulatory construct comprising:
a transposase sequence in reverse orientation downstream from a promoter sequence, which promoter sequence is controlled by an inducing agent;
a Cre recombinase sequence with expression controlled by the inducing agent; and,
wherein the transposase sequence is upstream from a reverse oriented first Lox sequence and downstream from a second Lox sequence, which second lox sequence is forward oriented between the promoter sequence and the transposase sequence; and,
wherein the stop sequence is bracketed by a pair of forward oriented lox sequences;
2) providing an expression transposon nucleic acid construct comprising:
a transposon sequence encoding a protein sequence of interest flanked by inverted terminal repeats (ITRs), which ITRs are the binding target of the transposase; and,
a binding site (BS) for the first transcriptional activator upstream from the ITRs;
3) applying the inducing agent to the cell, thereby inducing expression of Cre;
4) inverting the transposase to forward orientation by a CRE/Lox inversion, thus allowing expression of the transposase;
5) inserting the transposon at one or more safe harbor loci (SHL); and,
6) expressing the protein of interest in the host cell from the one or more SHL sites.

14. The method of claim 13, wherein the promoter sequence is a TRE3G promoter and the inducing agent is Dox/rtTA.

15. The method of claim 13, further comprising controlling a copy number of inserted transposons by adjusting the concentration of the inducing agent.

16. The method of claim 13, further comprising selecting host cells for blasticidin (Bsd) or II puromycin (Puro) resistance.

17. The method of claim 13, further comprising enabling the insertion of the transposon by knock-in into the SHL by AAV and CRISPR enzymes Cas9, Cas12a (Cpf1), Cas12b, CasX, or CasY.

18. The method of claim 13, wherein the protein of interest is an antibody protein.

19. The method of claim 13, wherein the SHL is a SHL of Table 3.
